(19) United States
(12) Patent Application Publication
(10) Pub. No.: US 2007/0254276 A1
(43) Pub. Date: Nov. 1, 2007

(54) Method and system for measuring membrane potential based on fluorescence polarization

(75) Inventors: Mordechai Deutsch, Moshav Olesh (IL); Yaniv Namer, Eilat (IL)

Correspondence Address: Martin D. Moynihan;PRTSI, Inc., P.O. Box 16446, Arlington, VA, 22215, US

(73) Assignee: Seng Enterprises Ltd., Larnaca (CY)

(21) Appl. No.: 11/592,956

(22) Filed: Nov. 6, 2006

Related U.S. Application Data
(60) Provisional application No. 60/834,789, filed on Aug. 2, 2006.
(60) Provisional application No. 60/794,878, filed on Apr. 26, 2006.

(51) Int. Cl. C12Q 1/00; G06F 19/00

(52) U.S. Cl. 435/4; 702/19

(57) ABSTRACT

A method of determining a membrane potential is disclosed. The method comprises (a) determining a difference in fluorescence polarization of a charged fluorescent probe being distributed across the membrane; and (b) determining a potential of the membrane, wherein the potential is proportional to the difference in the fluorescent polarization.

### RELATED APPLICATIONS

This Application claims the benefit of U.S. Provisional Patent Application No. 60/834,789 filed on Aug. 2, 2006, and U.S. Provisional Patent Application No. 60/794,878 filed on Apr. 26, 2006, the contents of which are hereby incorporated by reference.

### FIELD AND BACKGROUND OF THE INVENTION

The present invention relates to a method and system for measuring membrane potential.

The plasma membrane of a cell typically has a transmembrane potential of approximately −70 mV (negative inside) as a consequence of K⁺, Na⁺ and Cl⁻ concentration gradients that are maintained by active transport processes. Increases and decreases in membrane potential (referred to as membrane hyperpolarization and depolarization, respectively) play a central role in many physiological processes, including nerve-impulse propagation, muscle contraction, cell signaling and ion-channel gating. Potentiometric probes are important tools for studying these processes, and for cell-viability assessment.

Mitochondria inside the cell also comprise their own transmembrane potential. The mitochondrial respiratory chain produces energy which is stored as an electrochemical gradient which consists of a transmembrane electrical potential, negative inside of about 180-200 mV, and a proton gradient of about 1 unit; this energy is then able to drive the synthesis of ATP, a crucial molecule for a consistent variety of intracellular processes.

Measurement of the mitochondrial membrane potential provides the single most comprehensive reflection of mitochondrial bio-energetic function primarily because it directly depends on the proper integration of diverse metabolic pathways that converge at the mitochondria. Numerous diseases are associated with mitochondria dysfunction, including cancer, cardiovascular and liver diseases, degenerative and autoimmune disorders as well as aging and new pathologies related to mitochondria are identified each year.

Alterations in mitochondrial membrane potential is an important characteristic of a vast array of pathologies that either involve suppressed (e.g., cancer) or enhanced apoptosis (e.g., HIV, degenerative disease) as well as over 100 diseases directly caused by mitochondrial dysfunction such as DNA mutations and oxidative stress (e.g., various types of myopathies).

In general, there are two distinct methods to measure cell membrane potential, (a) direct electrical measurement of cell membrane potentials, e.g, the so-called ‘Patch Clamping’ technique (highly laborious and require very sophisticated technical skills), and (b) indirect optical sensing of both cell and mitochondrial membrane potentials using a membrane potential-sensitive dye as an indicator. The optical method that uses a fluorescent indicator has steadily gained popularity in recent years due to its convenience, high throughput and improved sensitivity.

Traditional optical measurements of membrane potential are based on the measurement of fluorescence intensity. However, intensity measurements are not relative and, consequently, are very sensitive to the measuring system and to background noises. In addition, it is unclear whether fluorescence is homogeneous in the cell (e.g. in the cytoplasm as opposed to the mitochondria). Thus, the relative intensities between different regions do not necessarily reflect the dye concentration ratio in them. Furthermore, there is the dependence of emission and excitation spectrums on chemo-physical aspects, in different regions in the cell, resulting in corresponding influences on the emitted intensities.

An alternative tool for analyzing the differential aspects of the fluorescence signal is fluorescence polarization (FP).

Fluorescence polarization was first described in 1926 by Perrin F., “Polarization de la lumiere de fluorescence vie moyenne des molecules dans l'etat excite”, J Phys Rad 7: 390-401 (1926). Fluorescence polarization measurements are based on the principle that a fluorescence labeled probe will emit fluorescence when excited by plane polarized light, having a degree of polarization inversely related to its rate of rotation. If the labeled molecule remains stationary throughout the excited state it will emit light in the same polarized plane; if it rotates while excited, the light emitted is in a different plane. Polarization is related to the time it takes a fluorescence labeled molecule to rotate through an angle of approximately 68.5 degrees: designated the correlation time.

Because polarization is a general property of fluorescent molecules, polarization-based readouts are somewhat less dye-dependent and less susceptible to environmental interferences such as pH changes than assays based on fluorescence intensity measurements.

Fluorescence polarization measurements have long been a valuable biophysical research tool for investigating processes such as membrane lipid mobility, myosin reorientation and protein-protein interactions at the molecular level.

Despite its widespread use, fluorescence polarity measurements have never been used to determine absolute values of membrane potentials since a linear relationship between the two has never been realized.

There is thus a widely recognized need for, and it would be highly advantageous to have, methods of determining membrane potentials devoid of the above limitations.

### SUMMARY OF THE INVENTION

According to one aspect of the present invention there is provided a method of determining a membrane potential, the method comprising: (a) determining a difference in fluorescence polarization of a charged fluorescent probe being distributed across the membrane; and (b) determining a potential of the membrane, wherein the potential is proportional to the difference in the fluorescent polarization.

According to another aspect of the present invention there is provided a system for determining a membrane potential comprising a processing unit, the processing unit executing a software application configured for converting a difference in fluorescence polarization of a charged fluorescent probe being in a distribution across the membrane to a membrane potential.

According to yet another aspect of the present invention there is provided a method of identifying an agent capable of altering a potential of a membrane, the method comprising: (a) contacting the membrane with the agent; and subsequently (b) determining an alteration in the potential of the membrane by (i) determining a difference in fluorescence polarization of a charged fluorescent probe being distributed across the membrane; and (ii) determining a potential of the membrane, wherein the potential is linearly proportional to the difference in the fluorescent polarization, wherein a change in the potential following step (b) as compared to a potential of the membrane untreated with the agent is indicative of an agent capable of altering a potential of the membrane.

According to still further features in preferred embodiments of the invention described below, the potential is linearly proportional to the difference in the fluorescent polarization.

According to still further features in preferred embodiments of the invention described below, the membrane is a naturally occurring lipid membrane.

According to still further features in the described preferred embodiments, the membrane is a synthetic membrane.

According to still further features in the described preferred embodiments, the membrane is a cell membrane or an organelle membrane.

According to still further features in the described preferred embodiments the membrane is a eukaryotic membrane.

According to still further features in the described preferred embodiments the membrane is a bacterial or viral membrane.

According to still further features in the described preferred embodiments, the eukaryotic membrane is selected from the group consisting of a mammalian membrane, a plant membrane and a fungal membrane.

According to still further features in the described preferred embodiments, the organelle membrane is selected from the group consisting of a mitochondrial membrane, a Golgi membrane and a nuclear membrane.

According to still further features in the described preferred embodiments, the synthetic membrane is a liposome membrane.

According to still further features in the described preferred embodiments, the plant membrane is a plastid membrane.

According to still further features in the described preferred embodiments, the plastid membrane is a chloroplast or a leucoplast membrane.

According to still further features in the described preferred embodiments, the membrane is intact.

According to still further features in the described preferred embodiments, the distribution is a steady-state distribution.

According to still further features in the described preferred embodiments, step (a) is effected by generating a map of fluorescence polarization for the distribution of the charged fluorescent probe across the membrane. According to still further features in the described preferred embodiments, the generating a map is effected by capturing an image of the distribution of the charged fluorescent probe across the membrane and calculating the fluorescence polarization for each picture element of the image.

According to still further features in the described preferred embodiments, the charged fluorescent probe is a cationic dye.

According to still further features in the described preferred embodiments, the charged fluorescent probe is an anionic dye.

According to still further features in the described preferred embodiments, the cationic dye is selected from the group consisting of a Blue-fluorescent SYTO dye, a Green-fluorescent SYTO Dye, an Orange-fluorescent SYTO dye, a Red-fluorescent SYTO dye, SYTO 62, Pur-1, thiazol, aryl, 2DS-7J1, Hoechst 33258, Hoechst 33342 and hexidium iodide.

According to still further features in the described preferred embodiments, the anionic dye is selected from the group consisting of an anionic a bis-isoxazolone oxonol dye, a bis-oxonol dye, Oxonol V, Oxonol VI, DiBAC₄, and DiBAC₂.

According to still further features in the described preferred embodiments, the charged fluorescent probe comprises a fluorescent label selected from the group consisting of rhodamine, tetramethyl rhodamine, carboxytetramethylrhodamine, carboxy-X-rhodamine, fluorescein, fluorinated fluoresceins, fluoresceinamine, carboxyfluorescein, alpha-iodoacetamidofluorescein, 4′-aminomethylfluorescein, 4′-N-alkylaminomethylfluorescein, 5-aminomethylfluorescein, 6-aminomethylfluorescein, 2,4-dichloro-1,3,5-triazin-2-yl-aminofluorescein, 4-chloro-6-methoxy-1,3,5-triazln-2-yl-aminofluorescein, fluoresceinisothiocyanate, 4,4-difluor-4-bora-3a,4a-diaza-s-indacene and its derivatives and cyanine dyes.

According to still further features in the described preferred embodiments, the system further comprises an imaging system.

According to still further features in the described preferred embodiments, the imaging system comprises an image capture apparatus.

According to still further features in the described preferred embodiments, the image capture apparatus is capable of capturing an image of the charged fluorescent probe being in a distribution across the membrane.

According to still further features in the described preferred embodiments, the imaging system further comprises a data processor for calculating the fluorescence polarization for each picture element of the image.

According to still further features in the described preferred embodiments, the data processor is further capable of generating a map of fluorescence polarization for the charged fluorescent probe across the membrane.

According to still further features in the described preferred embodiments, the data processor is further capable of determining an average membrane potential of more than one membrane.

According to still further features in the described preferred embodiments, the image capture apparatus is a fluorescent image capture apparatus and a light image capture apparatus.

According to still further features in the described preferred embodiments, the contacting is direct.

According to still further features in the described preferred embodiments, the contacting is indirect.

According to still further features in the described preferred embodiments, the membrane is a cell membrane and/or a mitochondrial membrane.

The present invention successfully addresses the shortcomings of the presently known configurations by providing a novel method for determining membrane potential which is simple, accurate and inexpensive to perform.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-I are intensity maps (FIGS. 1A, 1D and 1G), polarization images (FIGS. 1B, 1E and 1H) and their sections (FIGS. 1C, 1F and 1I—along the white line) of U937 Cells, stained with Rh123. Top panel=control cells, Middle panel=Nigericin treated cells, Lower panel=CCCP treated cells. Subtitles located at the side of pictured sections indicate, according to their order, respectively: Mean=mean polarization along white line, SD=standard deviation, Sum=total values along the white line, Y_{MAX} and Y_{MIN}=extreme polarization values.

FIGS. 2A-B are bar graphs illustrating the processing of images of U937 cells stained with Rh123. The results are the mean of the light intensity and polarization values that were calculated from images of single cells in the population. In each experiment, an analysis was conducted with about 30 cells. In every image of each single cell, a mitochondrial (Mit) area and a cytoplasm (Cytoplasm) area was defined. The columns represent average values and deviation values from the standard differential values for each mitochondrial and cytoplasm region in all the cells. The stars represent significant statistical differences according to the T test.

FIGS. 3A-B are bar graphs illustrating the image analysis of cells stained with Rh123 prior to and following treatment with oxidative phosphorylation uncoupler, CCCP. The results are average values of light intensity and polarization which were calculated from the images of single cells in the environment. In each experiment an analysis was conducted for about 30 cells. In every image for each single cell the mitochondrial (Mit) and the cytoplasm (Cytoplasm) areas were designated. Fluorescence intensity and polarization values are relative, while the corresponding values of untreated cells were defined as 1. The values of the columns represent the value averages for light intensity and polarization, and the deviation values from the norm that match all the mitochondrial and cytoplasm areas in all the cells. The stars represent significant statistical differences according to the T test.

FIG. 4 is a flow chart for experiments for Rh123 solution in whole cell and ruptured cell suspensions to test the influence of CCCP and Nigericin.

FIG. 5 is a line graph illustrating the emission spectrum of R123, Blue line-Rh123 solution (5 μg/ml) in PBS, Red line-CCCP (5 μM) Yellow line-Nigericin (5 μg/ml).

FIGS. 6A-D are fluorescence images with corresponding line profiles of U937 Cells, stained with Rh123 following Nigericin treatment. Panel A=Control measurement, Panel C=fluorescence image following exposure to CCCP, Panel B and D=Intensity Image Sections.

FIG. 7 is a flow chart for spectroscopic measurement comparisons between suspensions of whole cells and captured cells following treatment in the presence and absence CCCP and Nigericin.

FIGS. 8A-B are normalized emission spectra of cells, prior to and following rupture, and prior to and following treatment with CCCP.

FIG. 9 is a bar graph illustrating the overall emission Intensity of Rh123 in the cells, in a suspending solution and in a solution of ruptured cells, before and after treatment with CCCP. The stars represent significant statistical differences based on the ANOVA test.

FIGS. 100A-B are line graphs illustrating emission spectrum of cells prior to and following rupturein the presence and absence of Nigericin. Emission Spectra are not normalized.

FIG. 11 is a bar graph illustrating the general emission intensity of Rh123 in cells, in a suspending solution and in ruptured cell suspensions, prior to and following treatment with Nigericin. The stars indicate significant statistical changes according to the ANOVA test.

FIGS. 12A-B are bar graphs of normalized columns (relative to intensity and polarization values of untreated cells) of intensity and polarization value averages of the whole cell and for the mitochondria and the cytoplasm, prior to and following suppression. Standard deviation values are designated by the bars. The stars represent the significant statistical differences according to the T test.

FIGS. 13A-B are bar graphs of normalized columns (in relation to intensity and polarization values of untreated cells) of intensity and polarization value averages of the whole cell, the cytoplasm and the mitochondria, prior to and following treatment with CCCP. Standard deviations are indicated by the bars. The stars represent significant statistical differences according to the T Test.

FIGS. 14A-Y are confocal microscopy images illustrating sample planes. The height of the planes was measured from the bottom plane which is 8.4 micrometers lower than the focal plane. The mitochondria are the lighter areas in the diagram.

FIGS. 15A-P are fully illuminated deconvoluted microscopy images of U937 Cell Sections stained with Rh123. FIGS. 15A, C, E, G, I, K, M and O are images obtained prior to reconstruction and FIGS. 15 B, D, F, H, J, L and N are images obtained following reconstruction.

FIGS. 16A-G are fluorescence and computer generated images illustrating the reconstitution of fluorescent beads (FIGS. 16A-D) and mitochondria (FIGS. 16E-G). The central sample plane is illustrated prior to (16E) and following (16F) reconstruction. In close proximity to FIGS. 16E and F are pertaining lateral sections (thickness), below—plane Z-X, to the right Y-Z. FIG. 16G is a three-dimensional reconstitution of mitochondrial volume. Red arrows point to these sites in panel's 16F and 16G. Bar: 10 μm.

FIGS. 17A-B are semi-logarithmic graphs illustrating fluorescence intensity dependence (purple line-left ordinate) and it's polarization (black line-right ordinate) in concentration (FIG. 17A) and the average distance between two Rh123 molecules (FIG. 17B).

FIG. 18 is a schematic illustration depicting one embodiment of the system for determining a membrane potential of the present invention.

FIG. 19 is a photomicrograph of a typical cell.

FIG. 20 is a graph of fluorescent lifetime and fluorescent polarization as a function of the distance between two Rh123 molecules illustrating the short dynamic region associated with lifetime measurements.

### DESCRIPTION OF THE PREFERRED EMBODIMENTS

The present invention is of methods and systems which can be used to measure membrane potential.

Specifically, the present invention can be used in medical and biological research and to identify agents capable of altering membrane potential.

The principles and operation of the methods and systems according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Depolarization of membranes is an integral component of many signal transduction cascades. Abnormal changes at this level of signaling can be symptomatic of various pathological conditions. In epilepsy, for example, there are changes in the pattern of nerve activity in the brain preceding and following seizures. In cardiovascular systems irregular electrical activity can indicate underlying disease states. In the visual system, lack of activity in the retina indicates dysfunction in the detection of light.

Alterations in mitochondrial membrane potential is an important characteristic of a vast array of pathologies that either involve suppressed (e.g., cancer) or enhanced apoptosis (e.g., HIV, degenerative disease) as well as over 100 diseases directly caused by mitochondrial dysfunction such as DNA mutations and oxidative stress (e.g., various types of myopathies).

In addition measurement of membrane potentials across synthetic membranes such as liposomes may be important in determining drug delivery mechanisms.

The ability to accurately measure membrane potential therefore is of use in a wide range of fields.

Traditional optical measurements of membrane potential are based on the measurement of fluorescence intensity. However, intensity measurements are not relative and, consequently, are very sensitive to the measuring system and to background noises. In addition, it is unclear whether fluorescence is homogeneous in the cell (e.g. in the cytoplasm as opposed to the mitochondria). Thus, the relative intensities between different regions do not necessarily reflect the dye concentration ratio in them. Furthermore, there is the dependence of emission and excitation spectrums on chemo-physical aspects, in different regions in the cell, resulting in corresponding influences on the emitted intensities.

Whilst reducing the present invention to practice, the present inventors have uncovered using mathematical calculations a direct relationship between membrane potential and fluorescent polarization of a probe distributed across a membrane.

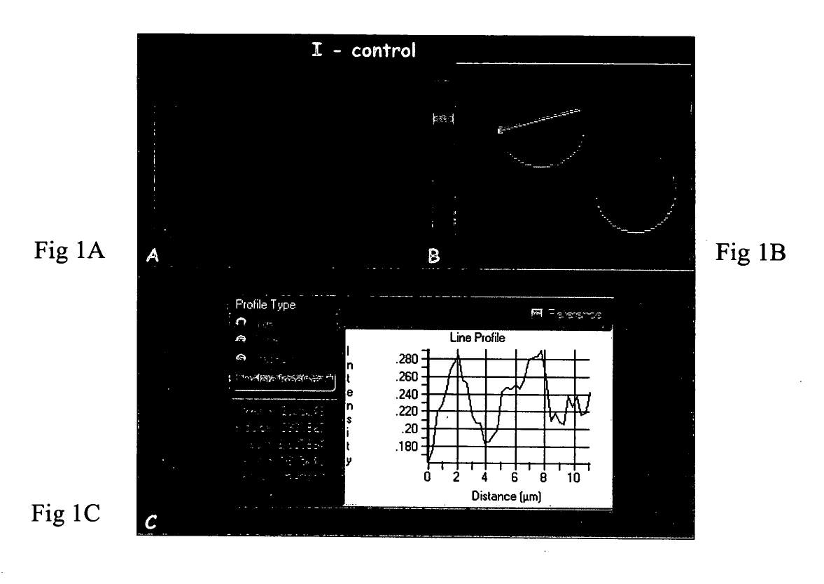

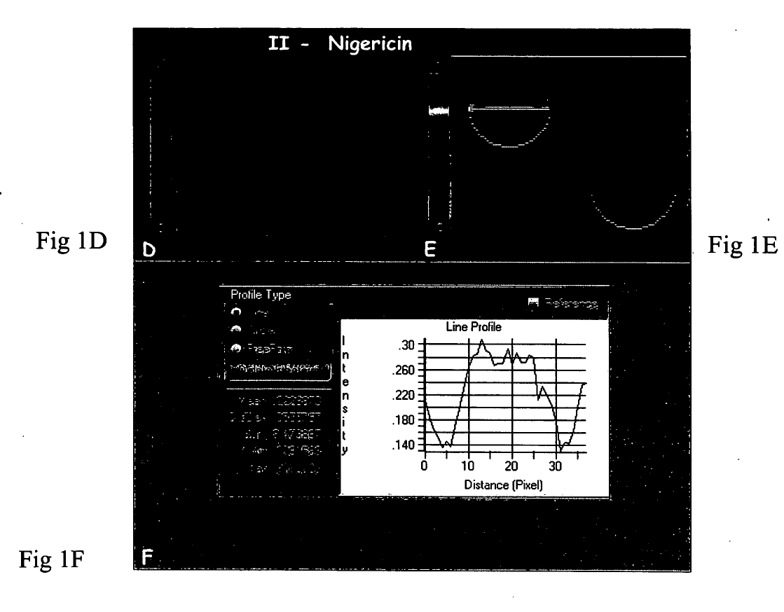

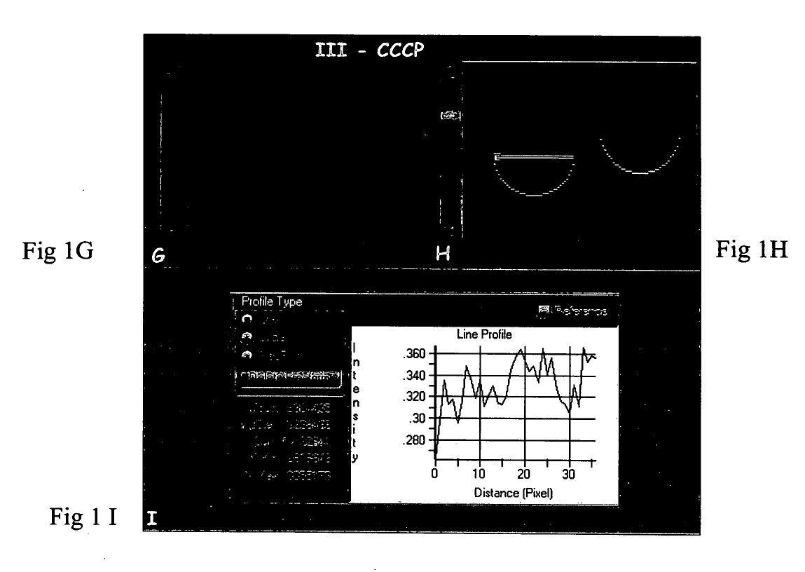

Accordingly, the present inventors have shown that manipulation of the Nernst equation leads to the novel supposition that membrane potential is proportional (e.g. linearly) to the change in fluorescent polarization of a probe on the inner and outer surface of a membrane. This relationship was verified by generation of polarization maps of a fluorescent probe across mitochondrial membranes in order to obtain an average fluorescent potential on both the inside and outside of a membrane—see FIGS. 1A-I.

Furthermore, this relationship was shown to hold true under conditions which are known to physiologically alter mitochondrial potential—see Example 5.

The present invention therefore utilizes a novel method to measure membrane potential that is simple, accurate and cheap to perform as it does not require the use of elaborate instrumentation.

Thus, according to one aspect of the present invention, there is provided a method of determining a membrane potential. The method comprises (a) determining a difference in fluorescence polarization of a charged fluorescent probe being in a distribution across the membrane; and (b) determining a potential of the membrane, wherein the potential is proportional to the difference in the fluorescent polarization.

As used herein, the phrase “membrane potential” refers to a difference in the electrical potential across a membrane. In the context of the present invention, such differences reflect transmembrane differences in the concentrations of charged molecules, such as sodium, potassium, and, particularly in the case of mitochondrial membranes, protons. It will be appreciated that a change in membrane potential may be due to a change in the environment or a change in ion permeability (e.g., consequence of membrane perfusion or activity of membrane proteins acting to actively transport or transfer ions across the membrane) of the membrane.

As used herein the term “membrane” refers to a synthetic or naturally occurring structure capable of passive or active ion transport.

Any membrane may be analyzed according to this aspect of the present invention so as to determine its membrane potential so long as it is in a state that it is accessible to a fluorescent probe and its environment may be imaged as further described below. According to a preferred embodiment of the present invention, the membrane is intact, although it is envisaged that a non-enclosed membrane may be analyzed such as in patch-clamping.

Thus, membranes of this aspect of the present invention comprise both naturally occurring lipid membranes (i.e. biological membranes such as cell membranes and organelle membranes) and synthetic membranes such as liposomes. A cellular sample being analyzed may be homogeneous, i.e. comprise an identical cell type or heterogeneous i.e. comprise non-identical cell types.

According to an embodiment of this aspect of the present invention, the membranes may be from a eukaryotic source. Examples of eukaryotic membranes include, but are not limited to mammalian membranes, plant membranes and fungal membranes.

According to another embodiment of this aspect of the present invention, the membranes are prokaryotic membranes (e.g., bacterial membranes) or viral membranes.

As mentioned hereinabove, the membrane potential of organelles may also be determined according to the method of the present invention. Such organelles include, but are not limited to mitochondria, Golgi bodies and a nuclei.

Exemplary plant membranes are plastid membranes such as chloroplast or leucoplast membranes.

The organelles may be situated inside the cell when being analyzed. Alternatively, organelles may be isolated from the cells and analyzed in an isolated state.

The term “fluorescence polarization” or “FP” as used herein refers to the effect in which visible or ultraviolet light is polarized with a filter and shines on part of a molecule, the fluorescent probe, which in turn fluoresces, emitting light of a longer wavelength than the original light.

Staining with a charged fluorescent probe may be effected by incubation for at an amount of time which is dependent on the probe. For example, the incubation time for Rh123 is typically 30 minutes.

Measurement of fluorescence potential requires excitation by polarized light which can be obtained using a laser or through light selection using a polarizing filter which only transmits light traveling in a single plane. Light of a defined wavelength may be refined by passing the light through a polarizing filter to obtain monochromatic, plane polarized light for excitation (for the present purposes designated “vertically polarized light”). Concurrently, the light path for collecting the emission light is modified by selective introduction of one of a pair of polarizing filters, one of which can be rotated to a position vertical to the plane of the excitation light, whereas the other of which can be rotated to a position horizontal to the plane of the excitation light. When a fluorescent sample in solution is introduced into the light path between the polarizer and the analyzers, only those molecules which are oriented properly to the vertically polarized plane absorbed light, become excited, and subsequently emit light. By selecting the appropriate analyzing filter to be inserted into the emission light path, the amount of emitted light in the vertical and horizontal planes can be measured and used to assess the extent of rotation of a fluorescent probe across a membrane.

The absolute fluorescent light polarization is calculated based on Perrin's formula as set forth below (Formula 1):

Polarization(P)=(Iᵥ−Iₕ)/(Iᵥ+Iₕ)

where Iᵥ is the intensity parallel to the excitation plane and Iₕ is the emission perpendicular to the excitation plane.

Instruments for measuring fluorescence polarization of a fluorescent agent are commercially available from such Companies as Invitrogen and Farrand Optical Components & Instruments.

An exemplary method for measuring fluorescence polarization is described hereinbelow.

A “fluorescent probe” as used herein refers to any compound with the ability to emit light of a certain wavelength when activated by light of another wavelength.

As used herein, the phrase “a charged fluorescent probe” refers to a potentiometric fluorescent probe (i.e. one whose distribution or chemical properties is effected by a change in electric potential) including, but not limited to cationic or zwitterionic styryl fluorescent probes, cationic rhodamines, anionic oxonols, hybrid oxonols and merocyanine 540. The charged fluorescent probe comprises a charged moiety which allows sensitivity to membrane potential and a fluorescent moiety which allows detection.

Any potentiometric probe may be used according to this aspect of the present invention as long as the change in the electronic structure of the probe or the distribution of the probe translates into a change in fluorescence polarization of the probe.

Since fluorescence polarization is not dependent on the intensity of the emitted light or on the concentration of the fluorophore, a change in fluorescence polarization of a probe whose distribution has been altered may for example be due to binding to intracellular or intravesicular sites, a change in the physical conditions of the environment or aggregation of the probe. The present inventors have shown through meticulous experimentation, that the change in fluorescence polarization of rhodamine under experimental conditions which change the mitochondrial potential are probably not a result of a change in viscosity or pH, but rather via a self-depolarization mechanism—see Example 3D and 3F.

An exemplary charged fluorescent probe that may be used according to this aspect of the present invention is a cationic dye. Examples of cationic dyes include, but are not limited to Blue-fluorescent SYTO dye, a Green-fluorescent SYTO Dye, an Orange-fluorescent SYTO dye, a Red-fluorescent SYTO dye, SYTO 62, Pur-1, thiazol, aryl, 2DS-7J1, Hoechst 33258, Hoechst 33342 and hexidium iodide.

Another exemplary charged fluorescent probe that may be used according to this aspect of the present invention is an anionic dye. Examples of anionic dyes include, but are not limited to an anionic a bis-isoxazolone oxonol dye, a bis-oxonol dye, Oxonol V, Oxonol VI, DiBAC₄, and DiBAC₂.

Typically, the charged fluorescent probe of the present invention comprises a fluorescent label. Examples of such fluorescent labels include, but are not limited to rhodamine, tetramethyl rhodamine, carboxytetramethylrhodamine, carboxy-X-rhodamine, fluorescein, fluorinated fluoresceins, fluoresceinamine, carboxyfluorescein, alpha-iodoacetamidofluorescein, 4′-aminomethylfluorescein, 4′-N-alkylaminomethylfluorescein, 5-aminomethylfluorescein, 6-aminomethylfluorescein, 2,4-dichloro-1,3,5-triazin-2-yl-aminofluorescein, 4-chloro-6-methoxy-1,3,5-triazln-2-yl-aminofluorescein, fluoresceinisothiocyanate, 4,4-difluor-4-bora-3a,4a-diaza-s-indacene and its derivatives and cyanine dyes.

Other charged fluorescent probes which may be used according to this aspect of the present invention are detailed in, Molecular Probes 1999 and the reference cited therein; Plasek et al. (1996) “Indicators of Transmembrane potential: a Survey of Different Approaches to Probe Response Analysis.” J Photochem Photobiol; Loew (1994) “Characterization of Potentiometric Membrane Dyes.” Adv Chem Ser 235, 151 (1994); Wu and Cohen (1993) “Fast Multisite Optical Measurement of Transmembrane potential” Fluorescent and Luminescent Probes for Biological Activity, Mason, Ed., pp. 389-404; Loew (1993) “Potentiometric Membrane Dyes.” Fluorescent and Luminescent Probes for Biological Activity, Mason, Ed., pp. 150-160; Smith (1990) “Potential-Sensitive Molecular Probes in Membranes of Bioenergetic Relevance.” Biochim Biophys Acta 1016, 1; Gross and Loew (1989) “Fluorescent Indicators of Transmembrane potential: Microspectrofluorometry and Imaging.” Meth Cell Biol 30, 193; Freedman and Novak (1989) “Optical Measurement of Transmembrane potential in Cells, Organelles, and Vesicles” Meth Enzymol 172, 102 (1989); Wilson and Chused (1985) “Lymphocyte Transmembrane potential and Ca.sup.+2-Sensitive Potassium Channels Described by Oxonol Dye Fluorescence Measurements” Journal of Cellular Physiology 125:72-81; Epps et al. (1993) “Characterization of the Steady State and Dynamic Fluorescence Properties of the Potential Sensitive dye bis-(1,3-dibutylbarbituric acid) trimethine oxonol (DiBAC.sub.4(3) in model systems and cells” Chemistry of Physics and Lipids 69:137-150, and Tanner et al. (1993) “Flow Cytometric Analysis of Altered Mononuclear Cell Transmembrane potential Induced by Cyclosporin” Cytometry 14:59-69.

As mentioned hereinabove, the charged fluorescent probe of the present invention is in a distribution across the membrane. It should be appreciated that the probe may be in any distribution across the membrane e.g. an initial distribution immediately following addition of the fluorescent probe or a steady state distribution. Typical equilibration times to allow the probes to redistribute across the membrane following alteration of a transmembrane potential are about 10-15 minutes.

According to this aspect of the present invention, the membrane potential is determined by determining the difference of the fluorescence polarization of the probe on either side of the membrane.

The present inventors have uncovered that the membrane potential is proportional to the difference in fluorescence polarization across a membrane. The proportionality may be any proportionality—e.g. it may be proportional to the square of the difference or the inverse of the difference.

According to a particularly preferred embodiment of this aspect of the present invention the membrane potential is linearly proportional to the difference of the fluorescence polarization of the probe on either side of the membrane.

Thus, in this case, in order to determine the absolute values of membrane potential, the difference in fluorescence polarization is multiplied by a constant. The constant comprises the components K_{B} (the Boltzmann constant), T (the temperature in Kelvin), e (elementary charge) and a and is set forth as follows.

$$Δ Ψ = \frac{{K}_{B} T}{ea} ( {P}_{M} - {P}_{C} )$$

Setting the temperature at 300 K, confines K_{B}T/e to a constant and is equal to 26 mV.

The parameter “α” is constant for any given fluorescent probe and may be determined by plotting a semi logarithmic plot of fluorescence potential on the y axis against probe concentration on the x axis. “a” is the gradient of the straight line. See FIG. 17A.

The conversion from fluorescence polarization of a fluorescent probe distributed across a membrane to membrane potential may be effected manually or using a computer system comprising a processing unit executing a software application configured for converting a difference in fluorescence polarization of a charged fluorescent probe being in a distribution across the membrane to a membrane potential.

As used herein, the phrase “processing unit” refers to a data processor, e.g., a computer.

The software application can be embodied in a tangible medium such as, but not limited to, a floppy disk, a CD-ROM, a hard drive of a computer, and a memory medium (e.g., RAM, ROM, EEPROM, flash memory, etc.). The software can be run by loading the software into the execution memory of the processing unit, configuring the processing unit to act in accordance with the instructions of the software. All these operations are well-known to those skilled in the art of computer systems.

According to an embodiment of this aspect of the present invention, the system may further comprise an imaging system.

Such an imaging system typically comprises an illuminating device, which for the purposes of measuring fluorescence polarization is a wavelength specific light source (e.g., an ultraviolet light source and the like). The illumination means also comprises a monochromator and a polarizer to produce plane polarized light. The illuminating device may also comprise a white light source which may be required for delineating the borders of the sample being analyzed. For example if mitochondrial membrane potential is being analyzed, white light is required for delineating the cell boundaries which comprise the mitochondria.

The imaging system of the present invention further comprises an image capture apparatus. The image capture apparatus typically includes means to acquire the image (e.g. a CCD) by translating the light into electronic impulses and transmits the image to a display device. The image capture apparatus may also include means for magnifying the image (e.g., a microscope). The CCD may be any suitable photosensitive device, including but not limited to a photomultiplier tube (PMT), a phototransistor, or a photodiode.

The imaging system may further comprise a data processor supplemented by an algorithm which calculates the fluorescence polarization for each picture element of the image. The algorithm may further determine an average fluorescence polarization for a particular distribution of a fluorescent probe. For example, the algorithm may determine an average fluorescence polarization of a fluorescent probe inside a mitochondria of a cell and an average fluorescence polarization of a fluorescent probe outside the mitochondria of that cell.

It is appreciated that there are numerous averaging procedures, including, without limitation, simple arithmetic mean, weighted average and the like, and the scope of the term “average” is intended to include all such types of averaging.

In addition, the algorithm may determine an average membrane potential of more than one membrane e.g. so that an average membrane potential may be measured for a given physiological state.

Examples of picture elements include, but are not limited to a pixel or a group of pixels.

According to a preferred embodiment of this aspect of the present invention, the display device displays a map of fluorescence polarization for the charged fluorescent probe distributed across the membrane. The map may be calculated by an algorithm.

According to a preferred embodiment of this aspect of the present invention, the image capture apparatus is calibrated for capturing an image of the charged fluorescent probe being in a distribution across a membrane. The calibrations typically take into account the membrane sample (e.g. amount of sample, type of membrane sample (e.g. cell membrane or organelle membrane) and the particular fluorescent probe used.

FIG. 18 is a block diagram schematically illustrating a system 10 suitable for performing the conversion from fluorescence polarization, in various exemplary embodiments of the invention. System 10 comprises an imaging system 12 and a processing unit 14 which is configured for converting a difference in fluorescence polarization of a charged fluorescent probe being in a distribution across the membrane to a membrane potential.

Imaging system 12 comprises a light source 16, a monochromator 18 and a polarizer 20 which passes polarized monochromatic light of the desired wavelength to a light transfer module (LTM) 22. The LTM 22 directs the excitation light from monochromator 18 onto a sample 24 placed on a platform 32, which can be, for example, a cell suspension. Any resulting fluorescent or luminescent light emitted by the sample may be collected by LTM 22 and is operative to direct those wavelengths to the entrance aperture of polarizer 26. In operation, polarizer 26 may be selectively adjusted to allow transmission of wavelengths from the emission spectrum of the fluorescent probe in the sample 24, and is operative to direct those wavelengths to an image capture apparatus 28, which detects the emitted light. The energy of the light can be determined based on the wavelengths which are already known from the selective adjustment. A data processor 30 communicating with image capture apparatus 28 receives imagery information provided thereby and generates a signal (which may be stored in a retrievable format for further analysis) indicating detection of the selected emission.

In general, all of the elements of the imaging system 12 in the device depicted in FIG. 18 including monochromator 18, polarizer 20 and the LTM 22, may be optically isolated in an optically isolated encapsulation coated internally with non-fluorescent absorptive material to minimize reflectance and light from other sources such as room light.

The method of determining membrane potential of the present invention may be particularly useful for assessing the effect of an agent (e.g. a pharmaceutical agent) on a membrane. Since changes in biological membrane potentials are associated with a wide range of diseases, the present invention may aid in the selection of a particular pharmaceutical agent for treating a disease.

In addition, the present invention may aid in diagnosis of a disease. Furthermore, the method of the present invention may be of use in medicinal and/or biological research shedding light on disease mechanisms and underlying causes.

Thus, according to another aspect of the present invention, there is provided a method of identifying an agent capable of altering a potential of a membrane, the method comprising: (a) contacting the membrane with the agent; and subsequently (b) determining an alteration in the potential of the membrane according to the method of the present invention, wherein a change in the potential following step (b) as compared to a potential of the membrane untreated with the agent is indicative of an agent capable of altering a potential of the membrane. According to this aspect of the present invention, the membrane is contacted with an agent for a sufficient time for the agent to produce a biological effect.

It will be appreciated that the contacting may be direct or indirect. For example, the agent may be administered to an area other than the membrane which is to be analyzed. The change in membrane potential may then occur as a knock-on effect. The effect may be immediate or delayed such that the change in membrane potential may be monitored in real time.

According to a preferred embodiment of this aspect of the present invention, the membrane is a biological membrane (e.g., a cell). The membrane may be derived from a healthy subject or may be from a subject suffering from a disease or disorder. Alternatively, the membrane may be derived from an animal which has been adapted to act as an animal model of a disease.

According to this aspect of the present invention, the membrane potential of the sample being analyzed is known prior to contacting with the agent. This knowledge may be based on literature or supposition. Alternatively, or additionally, this knowledge may be based on measurements effected prior to contacting with the agent. Such measurements may comprise the membrane potential determining methods described in the instant application or any other form of membrane potential determining methods.

As mentioned, following identifying an agent capable of altering a potential of a membrane, such an agent may be selected as a drug candidate for treating a particular disease. Thus, for example an agent which shows that it is effective at changing mitochondrial potential may be considered a candidate for treating a mitochondrial disease.

Exemplary mitochondrial diseases or disorders include, but are not limited to MELAS, LHON, MERRF, MNGIE, NARP, PEO, Leigh's Disease, and Keams-Sayres Syndrome.

Although the primary function of mitochondria is to convert organic materials into cellular energy in the form of ATP, mitochondria also play an important role in many metabolic tasks, such as apoptosis, glutamate-mediated excitotoxic neuronal injury, cellular proliferation, regulation of the cellular redox state, heme synthesis, steroid synthesis and heat production (enabling the organism to stay warm). Thus agents which show that they are effective at changing mitochondrial potential may be considered candidates for treating diseases or disorders which are related to these phenomena.

Mitochondrial dysfunction also contributes to cell death during the acute pathology of stroke and myocardial infarction, and it is increasingly implicated in the etiology of chronic degenerative disorders such as Alzheimer's and Parkinson's diseases, as well as type 2 diabetes.

Conversely, agents which have been shown to change mitochondrial potential may be used to intentionally undermining mitochondrial stability (as reflected by its membrane potential) in order to induce necrosis or apoptosis and may provide novel avenues for development of anti-tumor agents.

As used herein the term “about” refers to +10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Fluorescence Polarization measurements in U937 Cells Stained with Rh123

Fluorescence polarization (FP) evaluation was conducted using U937 promonocytic cells under the following physiological conditions: control, following treatment with CCCP and following treatment with Nigericin. All the cells were stained with Rh123, rinsed, loaded in cell chips and then measured.

Materials and Methods

Cell treatment: U937 promonocytic cells were treated with an oxidative phosphorylation uncoupler CCCP (commercially available from Sigma-Aldrich) at a concentration of 5 μM and Nigericin (commercially available from Sigma-Aldrich) at 5 μg/ml for 15 minutes.

Cell staining: Cells were stained by treating with 3-13 μM Rh123 and loaded in cell chips.

Fluorescence polarization measurement: Transmitted light images and fluorescence images were obtained parallel to the excitation field (excitation and emission polarizers set at 0°), and perpendicular to it, with the excitation polarizer set at 0°, and the emission polarizer set at 90°. From every pair of parallel and perpendicular images, the FP value was calculated for each pixel, using the following equation and a polarization map was created according to the following equation:

$$Equation 1 \text{:}$$

$$P = \frac{{I}_{II} - M \cdot {I}_{⊥}}{{I}_{II} + M \cdot {I}_{⊥}}$$

In order to obtain a calibration reference point, the polarization value was obtained from the entire cell. This is the only measurement that is comparable to parallel microscopic measurements. As the fluorescent dye does not stain the whole cell, it is not possible to obtain the cell size and/or identify from where the fluorescence was emitted in order to calculate the average polarization. Accordingly, a cell chip was used which allowed defining the borders of the cell by means of images of the cell received through transmitted light photographs. The borders were projected on the polarization map and the FP average was calculated for each cell. This process was implemented to evaluate all the cells, and a general average was obtained for each given physiological state.

Results

FIGS. 1A-I show polarization and intensity maps of the control cells, as well as, the cells treated with CCCP and Nigericin.

The overall polarization values that were calculated from the entire cell in the three different situations are listed in Table 1, hereinbelow:

TABLE 1

Mean FP

| | Left Cell | Right Cell |
|---|---|---|
| Control | 0.268 ± 0.035 | 0.267 ± 0.032 |
| Nigericin | 0.255 ± 0.057 | 0.253 ± 0.048 |
| CCCP | 0.343 ± 0.034 | 0.324 ± 0.046 |

The spectrum of the polarization map illustrated in FIGS. 1A-I, indicates that the mitochondrial compartments (after being designated in the polarization map by projecting the lighter areas defined in the intensity image, see the red line) possess significantly lower polarization values in relation to their environment. Quantitative confirmation for this observation is found in the polarization sections, respectively.

A similar test of the same map, after demarcation, indicates a significant rise in polarization in the mitochondrial area, along with a reduction in their intensities. Testing the same particulars in cells treated with Nigericin (FIGS. 1D-F) illustrates an “intensification” of the phenomena in the control experiments. Polarization values in the mitochondrial areas are lower, whereas, in other areas, they are higher.

The findings described above, were collected from many evaluations, from hundreds of single cells. It was found, that the affected changes are substantial, even on a mean level. The average results, based on 300 cells, in the three different conditions, are listed in Table 2, hereinbelow:

TABLE 2

| Mean Polarization | Treatment |
|---|---|
| 0.271 ± 0.023 | None |
| 0.258 ± 0.025 | Nigericin |
| 0.346 ± 0.013 | CCCP |

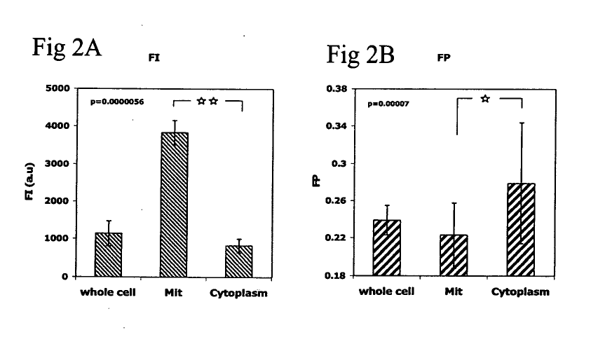

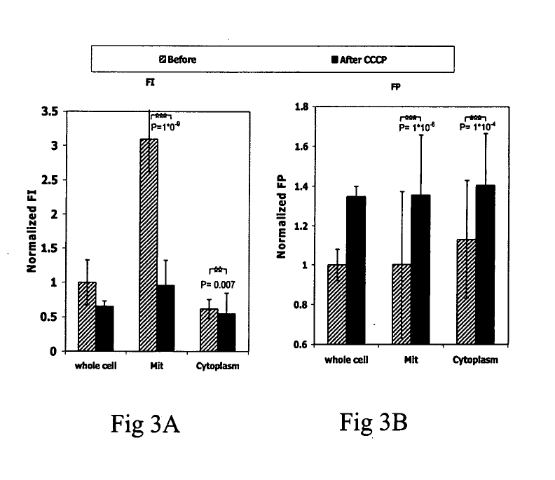

Analysis of the images illustrated in FIGS. 1A-C and 1G-I are presented in FIGS. 2A-B and 3A-B. These analyses show the differences between monitoring membrane potentials using FI and FP.

### Conclusion

The consistency of these findings, derived from many experiments, furnished the conclusion that, with a very high level of probability, fluorescence polarization reflect new aspects that, in general, were not tested until now, and in particular, relate to the mitochondrial potential in different physiological situations.

### Example 2

### Fluorescence Lifetime Measurements in U937 Cells Stained with Rh123

Fluorescence lifetime (FL) is essentially a differential parameter, implying, that if the emission is heterogeneous, it is obtained from a linear sum of pure decay components; therefore, analyzing results of FL measurements will probably indicate the existence of several probes in a sample, even in situations where the sample was stained with only one type of dye. This latter instance, probably, will happen when the probe, distributed in different cell regions, “senses” a different environment in terms of chemo-physical properties, that may possibly change the spectroscopic aspects of the marker and, among others, its' fluorescence lifetime. This situation, also, may be derived from the fact that the dye concentration in the cell is heterogeneous and, as such, tends to accumulate in certain areas more than others.

The distribution of decay components may also change as a result of treatment under varied physiological conditions, and is it possible to obtain information from the measurement data about the changes in the probe distribution through the procedure of “blind” measurements, i.e., without observing the sample being measured.

Materials and Methods

Cell treatment: U937 promonocytic cells were treated with an oxidative phosphorylation uncoupler CCCP and Nigericin as described hereinabove.

Cell staining: Cells were stained by treating with Rh123 and loaded in cell chips.

Fluorescence lifetime measurement: Measurements were carried out using polarizers that were set at magic 0.54°. Rh123 was excited by a 488 nm laser line. Fluorescence testing was conducted from 500 nm wavelengths and above (toward the red area).

Results

Lifetimes and decay components measured in cells stained with Rh-123 without any treatment and with treatment of CCCP and Nigericin are summarized in Table 3 hereinbelow.

TABLE 3

| f₂(weight) | α₂(decay) | τ₂(lifetime | f₁(weight) | α₁(decay) | τ₁(lifetime) | Treatment |
|---|---|---|---|---|---|---|
| 0.406 | 0.656 ± 0.047 | 2.088 ± 0.047 | 0.594 | 0.344 ± 0.029 | 5.395 ± 0.580 | Control |
| 0.133 | 0.310 ± 0.100 | 1.475 ± 0.423 | 0.867 | 0.690 ± 0.105 | 4.271 ± 0.292 | CCCP |
| 0.670 | 0.862 ± 0.030 | 2.519 ± 0.174 | 0.330 | 0.138 ± 0.048 | 7.735 ± 0.828 | Nigericin |

In the three kinds of measurements, the X² (Goodness-of-fit parameter by lest-squares analysis) value did not exceed 1.5. Table 3 shows only two decay components for each of the treatments; therefore, given that, in the images of intensity and polarization, one can see that the probe is found in two regions only (although, in different concentrations in the various treatment situations), the mitochondria and the cytoplasm; then, it is possible to assume, with a very high probability, that the two decay components which were found, reflect the characteristics of the probe in these different areas. Moreover, it appears from the weights of the decay components, it may be possible to gain insight into the quota of dye accumulated in the mitochondria, as opposed to the quota of dye accumulated in the cytoplasm, in each treatment. For this reason, measuring lifetime allows the unique possibility to investigate the changes in probe distribution in the cell by using “blind” measurement which produces differential information.

The results were obtained from an average, encompassing 30 evaluations, with standard deviations recorded accordingly. The outstanding result is the fact that two lifetime groups were obtained, one long—above 4 nanoseconds—and one short—below 3 nanoseconds. In order to determine which region correlates with which lifetime group both the lifetimes and the weights were considered.

With reference to the results obtained from treatment with CCCP, the value r is 4.27 nsec. This value is similar to the lifetime of Rh123 in a water suspension (PBS), which is over ˜4.2 nsec; furthermore, the weight of this component exceeded 70%, which suggests that most of the emission signal from the stained cell has its' source in the “watery” area. Connecting these findings to the fact that, with the induction of CCCP, the probe leaks from the mitochondria to the cytoplasm, which is an environment rich in water, it may be presumed that this measurement refers to a probe found in the cytoplasm.

Thus, the “short times,” indicated in Table 3, may be attributed to the probes found in the mitochondria. In addition, it has been corroborated that the lifetime of Rh123, as measured in single mitochondria, is 2 nsec in the control state.

In accordance with the above explanations, the following pattern emerges: in control measurements of U937 cells stained with the given Rh123 concentration, about 34% of the fluorescence signal is emitted from the molecules found in the cytoplasm and about 66% of the fluorescence signal is emitted from molecules found in the mitochondria. The relationship between these weights intensifies as a result of treatment with Nigericin, i.e. only 14% of the fluorescence signal is from the probe found in the cytoplasm and 86% from the probe located in the mitochondria. The two outcomes conform to the expectation that treatment with Nigericin increases the electric polarity of the mitochondria and, thus, increases the level of the dye in it.

Following induction with CCCP, the probe distribution changes and, primarily, the measured signal changes. The result being, about 70% of the signal originates from the probe in cytoplasm and about 30% of the signal comes from mitochondrial regions. It is interesting to note, there is a reverse symmetry between the control condition and the condition following treatment with CCCP; in other words, the weights of the signals from the different areas, reverse.

Close examination of Table 3 may also provide insights into understanding the source of the long lifetime—which is over 7 nsec.

Referring to Table 3, column τ₁, a trend of increasing lifetime, with a simultaneous decrease in the weight of the fluorescence signal from the cytoplasm (second and third columns) can be seen. “Natural lifetime” may be defined as the lifetime of a molecule that does not interact with its surroundings. The lifetime of this molecule may become longer as a result of intra-molecular passages that create phosphorescence (restricted emission), or alternatively, as a result of the molecule serving as an acceptor from a molecule having a long lifetime. The first case is not plausible, in that, there is only a slight possibility of getting Rh123 phosphorescence at room temperature.

In the second case, a process of energy transfer may occur, which shortens the lifetime of the donor, and in this specific situation, from a long unknown value down to 7 nsec. The emission takes place, but, through Rh123 molecules.

### Example 3

### Spectroscopic Aspects of Rh123 in U937 Cells in Isolated Mitochondria

The following example describes a series of experiments which were performed in order to understand the spectroscopic aspects of Rh123 in isolated mitochondria of U937 cells.

Firstly, the direct influence of CCCP and Nigericin on Rh123, were tested.

Secondly, the direct influence of changing factors in the cell during activation, for example, pH and viscosity, on the lifetime and/or polarization of Rh123, was tested.

Thirdly, spectroscopic properties of the whole cell and of a suspension of ruptured cells were tested, in order to acquire information about the influence of the whole cell on the intra-cellular marker, while under the influence of various treatments.

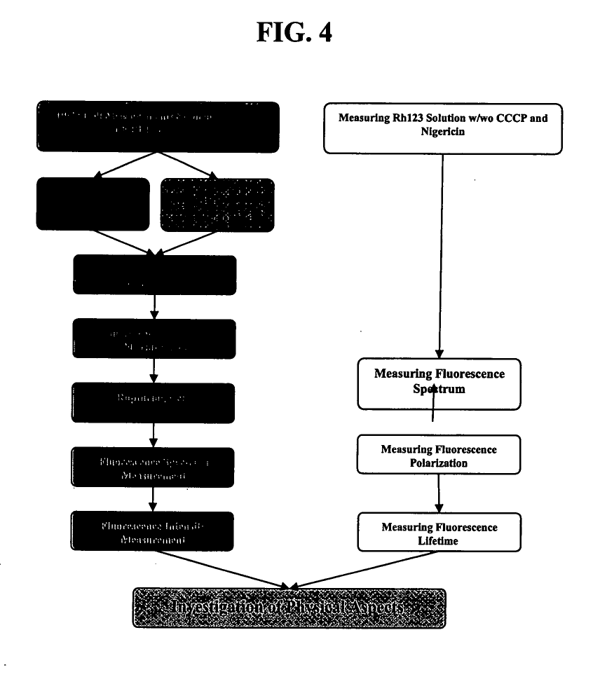

In order to distinguish between the cell's influence and the mitochondria's influence, the same experiments with isolated mitochondria were repeated. In all the experiments, the fluorescence intensity was calculated through integration over the spectrum graph. A flow chart of the sequence of all these experiments is illustrated in FIG. 4.

A. Influences on the Emission Spectrum:

Materials and Methods

A suspension of Rh123 at a concentration of 0.5 μg/ml was prepared. The suspension was divided into three aliquots, each containing a volume of 1 ml. To the first part was added 2.5 μl of PBS. To the second part was added 2.5 μl CCCP at a concentration of 2 mM, so that a final concentration of 5 μM CCCP was reached. To the third part was added 2.5 μl of Nigericin, at a concentration of 2 mg/ml, so that a final concentration of 5 μg/ml Nigericin was reached.

In the first stage, the emission spectrum of the suspension was obtained, using CARY ECLIPSE. The suspensions were excited with 480 nm light and the fluorescence was measured between 500-600 nm, while the slits (emission and excitation) were 2.5 nm.

Results

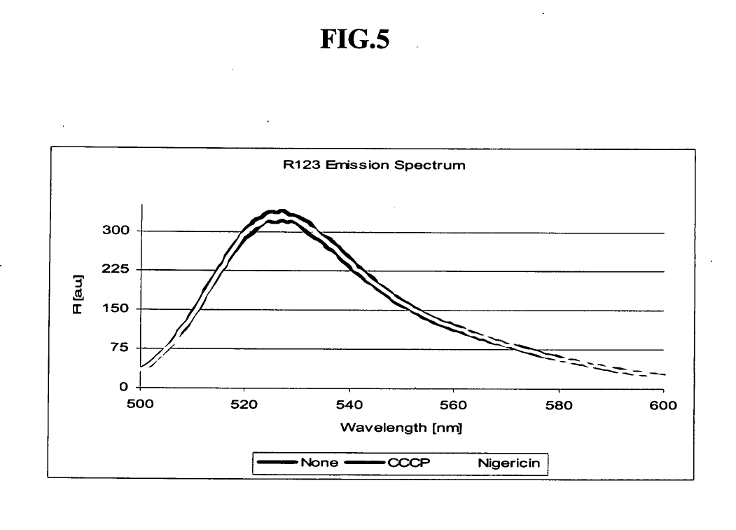

The measurement results are depicted in FIG. 5. The measurement results do not testify to the direct influence of CCCP and/or 5 μg/ml Nigericin on the emission spectrum of Rh123 under the experimental conditions.

B. Influences on Fluorescence Polarization and Lifetime in Suspensions:

The direct influence of CCCP and/or Nigericin were tested on the same suspensions of Rh123, measuring the lifetimes in contrast to the control.

Results

The results are recorded in Table 4 hereinbelow.

TABLE 4

| P | τ [nsec] | Treatment |
|---|---|---|
| 0.057 ± 0.005 | 4.236 ± 0.008 | None |
| 0.053 ± 0.001 | 4.241 ± 0.002 | CCCP |
| 0.056 ± 0.002 | 4.242 ± 0.004 | Nigericin |

It may be concluded that CCCP and Nigericin do not directly influence the lifetime nor the polarization of Rh123 in liquid suspensions.

C. Testing the Direct Influence of pH on the FLT of Rh123

The intra-cellular environment is heterogeneous and multi-phased; therefore, changes may occur in the acidity level in different regions in the cell. Measurements were executed, for this reason, to test if the acidic level has any influence on the fluorescence lifetime of the Rh123. The measurements were conducted in a K2 system, in a suspension of Rh123 with a concentration of 13 μM and the suspension of NAO, in a concentration of 10 μM with an acidity level of 6.0, 7.4 and 8.5.

### Results

The results are recorded in Table 5, hereinbelow:

TABLE 5

Fluorescence life-time τ_{F} [nsec]

| pH | Rh123 5 μg/ml | NAO 10 μM |
|---|---|---|
| 6.0 | 4.204 ± 0.064 | 1.086 ± 0.028 |
| 7.4 | 4.325 ± 0.007 | 1.051 ± 0.003 |
| 8.5 | 4.215 ± 0.037 | 1.066 ± 0.006 |

Table 5 indicates that the change in the acidity level of the environment in which the cell is hosted, does not influence the fluorescence lifetime.

D. Testing the Direct Influence of viscosity on the FLT and FP of Rh123

In these tests, the fluorescence lifetime τ_{F} was also measured along with the rotational decay time τ_{R}. For this, Rh123 (5 μg/ml) and suspension NAO (10 μM) were dissolved in three glycerol suspensions (30%, 60% and 80% v/v) in PBS. Each measurement was performed in triplicate.

Results

The results are recorded in Table 6, hereinbelow.

TABLE 6

| | Glycerin | | | |
|---|---|---|---|---|
| Dye | [%] | τ_{F} [nsec] | τ_{R} [nsec] | FP |
| NAO | 30 | 1.599 ± 0.072 | 0.923 ± 0.181 | 0.110 ± 0.007 |
| 10 μM | 60 | 2.081 ± 0.056 | 1.298 ± 0.057 | 0.179 ± 0.009 |
| | 80 | 2.556 ± 0.009 | 3.888 ± 0.181 | 0.303 ± 0.004 |
| RH123 | 30 | 3.861 ± 0.142 | 0.460 ± 0.010 | 0.090 ± 0.007 |
| 5 μg/ml | 60 | 3.536 ± 0.018 | 2.219 ± 0.006 | 0.200 ± 0.005 |
| | 80 | 3.500 ± 0.211 | 8.463 ± 0.047 | 0.366 ± 0.010 |

Table 6 shows that with the increase of the viscosity level from 30% to 80%, the lifetime of Rh123 slightly shortens although this effect is negligible when compared to FP or τ_{R}. In summary, it is not believed that the changes in the cell micro-viscosity can explain the differences between the polarizations and the lifetimes measured in the cytoplasm and in the mitochondria, prior to and following ionifration.

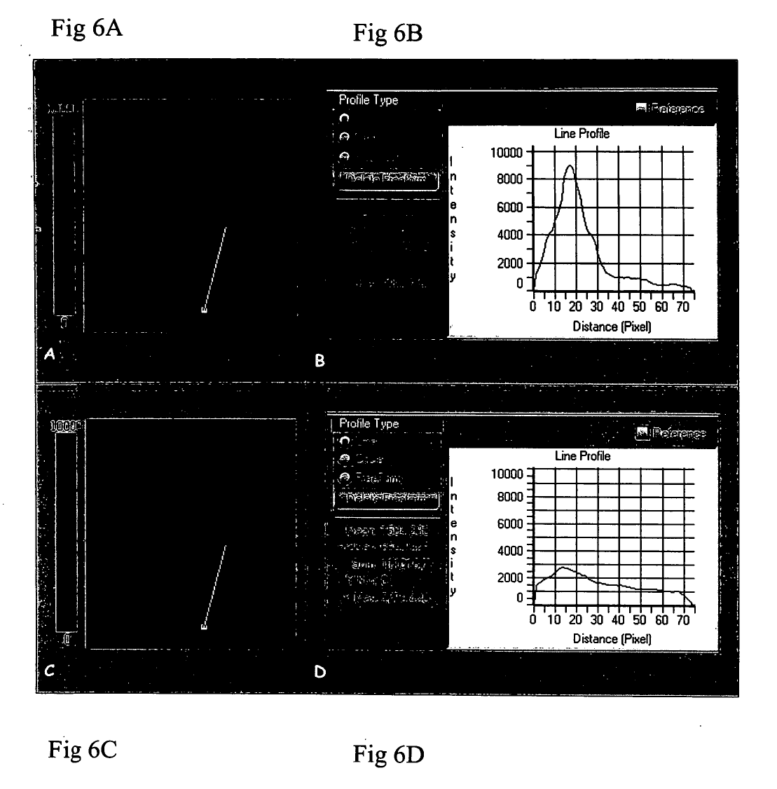

Even if the increase in NAO lifetime is affected by viscosity, it does not explain the increase in lifetime resulting from treatment with Nigericin. Additional examination of the polarization sections in FIG. 6 shows that following treatment with Nigericin, there is no significant change in the polarization measured in the cytoplasm, despite the lengthening of the lifetime and it would not be logical to relate the cell's lifetime elongation to the rise in viscosity, as in the case of NAO. Therefore, it may be assumed that an “unidentified molecule” that operates as a Donor for Rh123 acting as an Acceptor.

E. Spectroscopic Measurements in Whole and Ruptured Cells

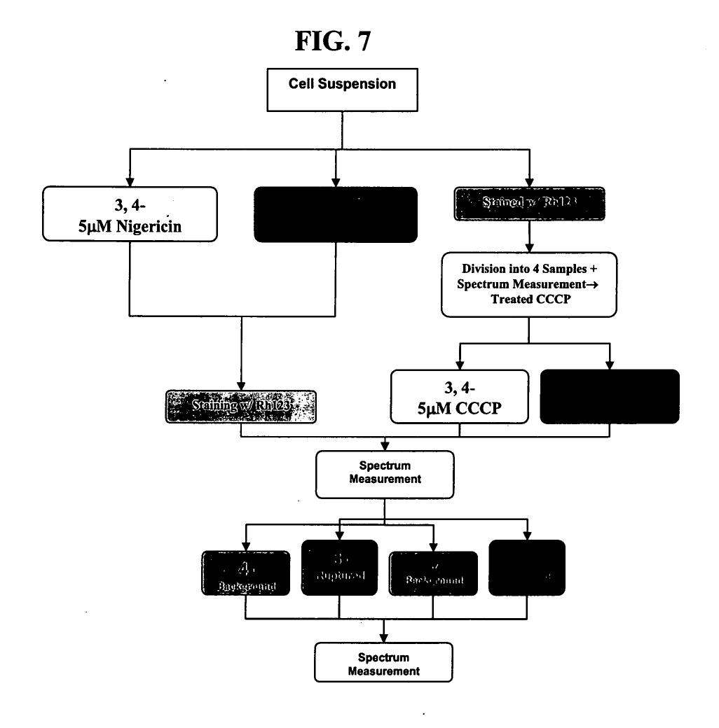

There were two main goals to these experiments. The first goal was to ascertain whether a fluorescence intensity decreases in cells treated with CCCP, and similarly, to ascertain whether fluorescence intensity increases in cells treated with Nigericin. The second goal was to learn about the characteristics and amount of influence of the cellular medium on the Rh123 spectroscopic properties. A flow diagram describing the sequence of the experiments is presented in FIG. 7.

E1: CCCP

Materials and Methods

The cells were stained with Rh123 at a concentration of 3 μM and suspended in PBS, at a cell concentration of 350,000/ml. At this cell concentration, no scattered signal was detected in the fluorescence measurement setup.

The measurement was conducted in CARY spectrofluorometer. The excitation was performed at 480±10 nm and the fluorescence was collected using a 530±10 nm filter. The cell suspension was divided into 4 aliquots in volumes of 2 ml, each. First, the spectrum for all the parts was measured and a comparison was performed to verify equal cell concentration in each quota. Second, parts 3 and 4 were treated with CCCP, while acetone was added to parts 1 and 2, at the same concentration and volume as that in which the CCCP was dissolved, and a spectrum measurement was made. At the end of this process, parts 1 and 3 were ruptured by sonication (VCX 130, Sonics and Materials Inc, Newtown, USA), and parts 2 and 4 were centrifuged and the supernatant was removed to measure the background. Rupturing the cells causes the complete release of the probe (microscopic tests did not reveal any cell fragments).

At the end of this process, the spectrum was measured.

Results

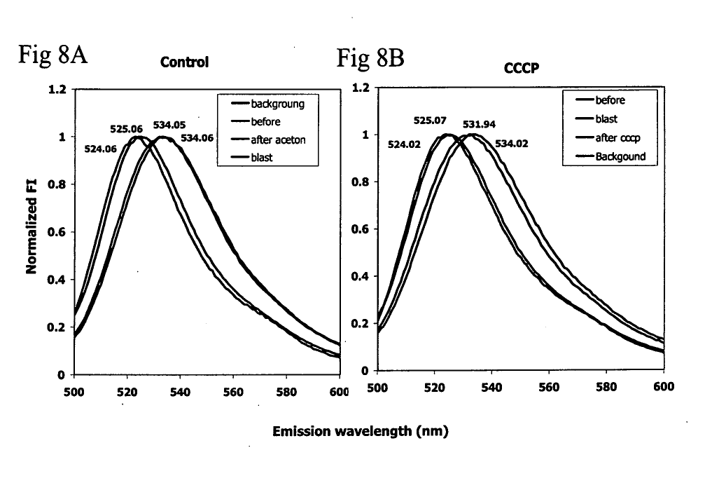

The results of these experiments are presented in FIGS. 8A-B.

The spectra were normalized to emphasize the differences between the spectra. It is possible to see that the fluorescence of intra-cellular Rh123 is shifted toward the red area, in relation to that which is free, by about 9 nm. This outcome is expected, although the degree of shifting is changeable, as it results from association of the probe with the proteinaceous environment. Of note, addition of acetone (the CCCP solvent) does not alter the spectrum (FIG. 8A, red line), even as regards the shift to the blue, following activation of CCCP (FIG. 8B, red line). Therefore, even though the latter change is, in total, from 534 to 532 nm, repeating the measurement, with and without acetone, illustrates that it is, indeed, significant. This finding repeated itself. Therefore, the spectrum, prior to stimulation with CCCP (part of the probe is located in the mitochondria) is the result of the dye's spectra in both the mitochondria and the cytoplasm. Following subsequent leakage of the probe from the mitochondria to the cytoplasm (after stimulation with CCCP), the maximum wavelength is shifted to the blue. Therefore, it can be deduced that only the probe spectrum in the mitochondria was shifted toward the red wavelengths beyond 534 nm.

Conclusion

These findings suggest that the marker comprises different spectroscopic properties in various regions.

In summary, it is possible to state that Rh123 in the mitochondria not only displays a different fluorescence lifetime and fluorescence polarization from the probe found in the cytoplasm, but, also a different emission spectrum.

Since there are differences in the emission spectrum of Rh123 in the mitochondria and cytoplasm, the area under the emission graphs must also be calculated in estimating the given spectral effect of CCCP. The area is proportionate to the number of emitting molecules and not to the measured intensity.

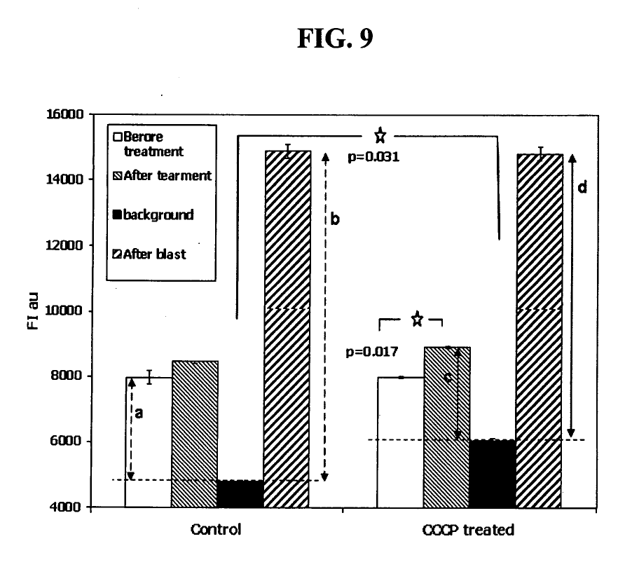

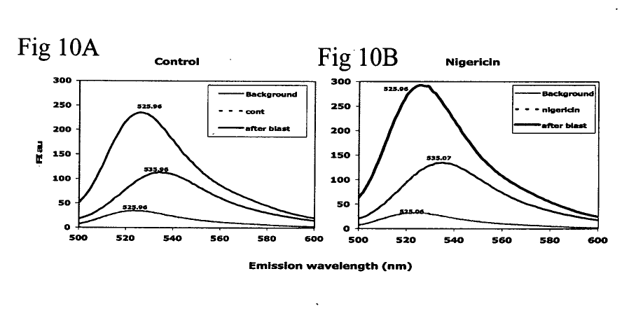

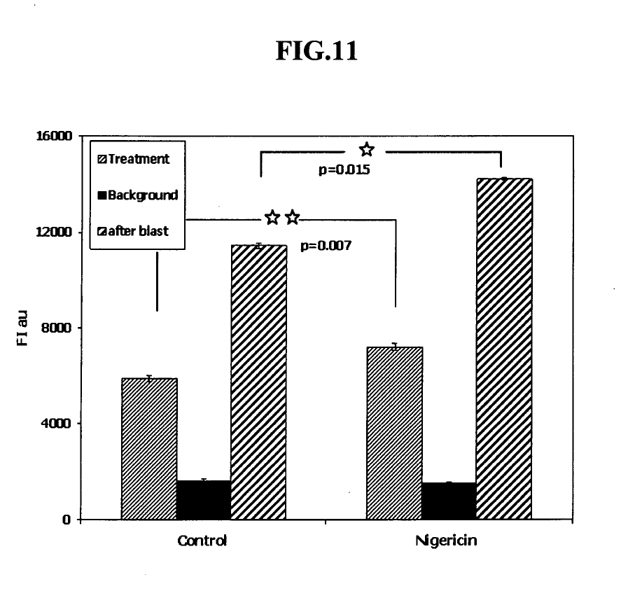

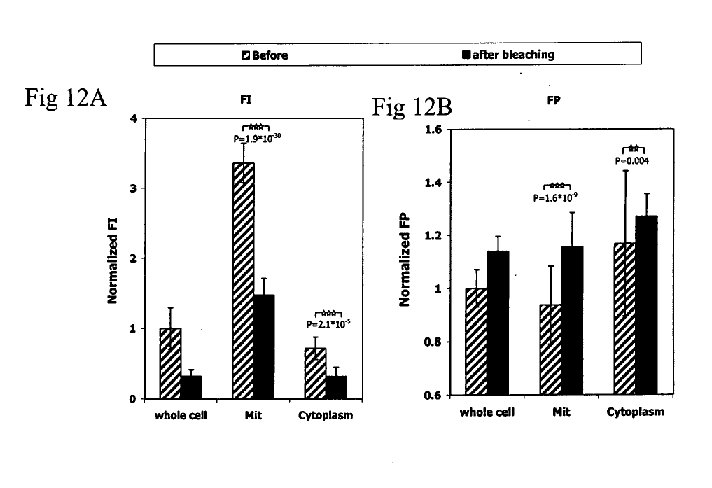

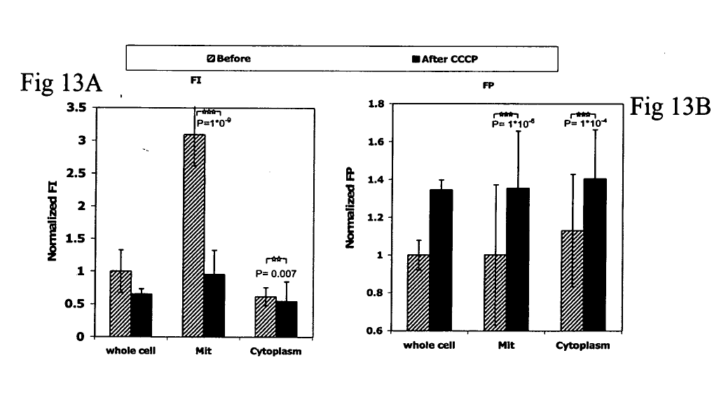

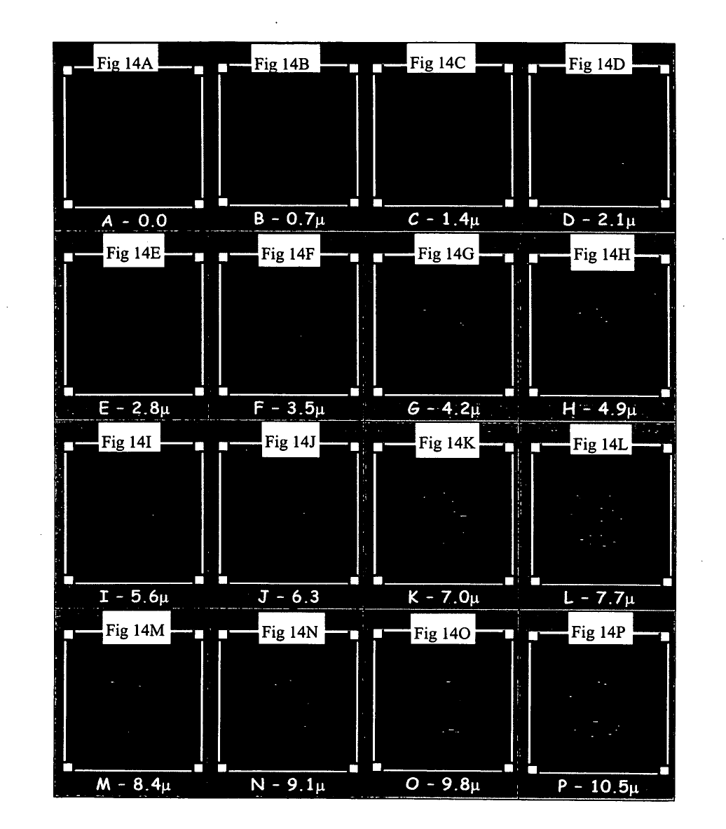

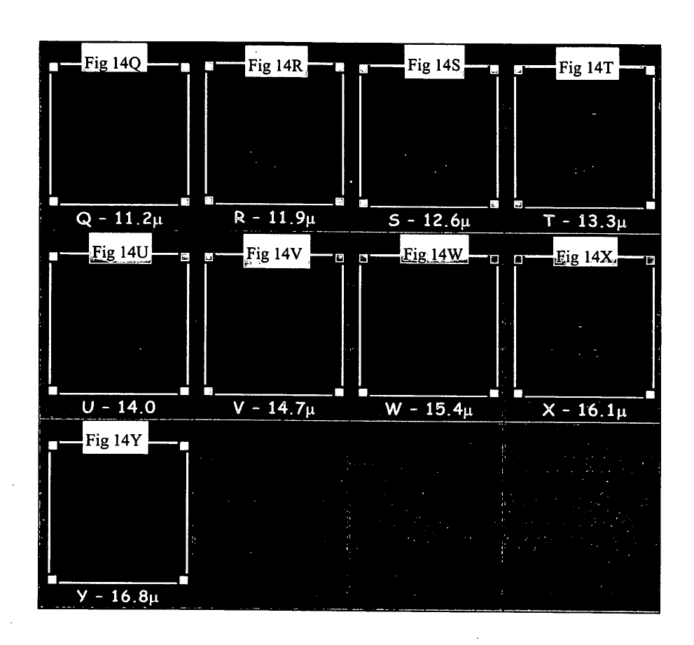

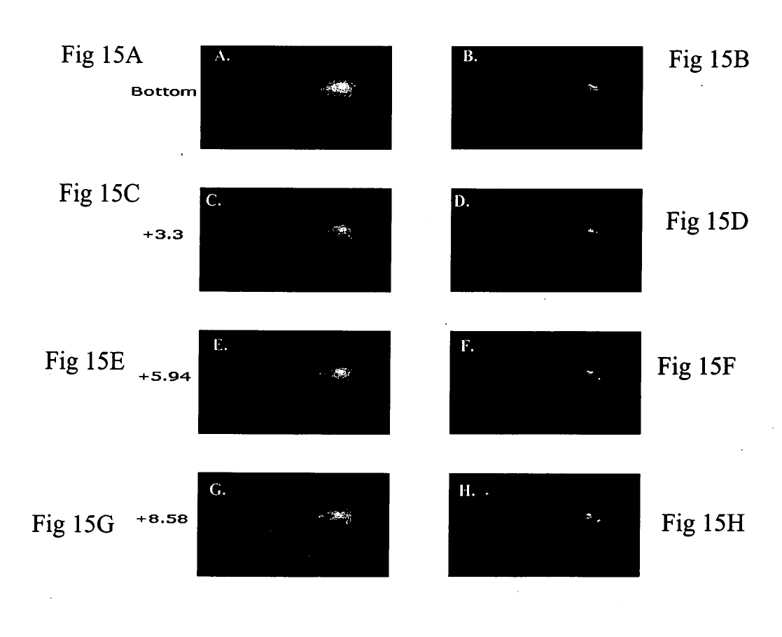

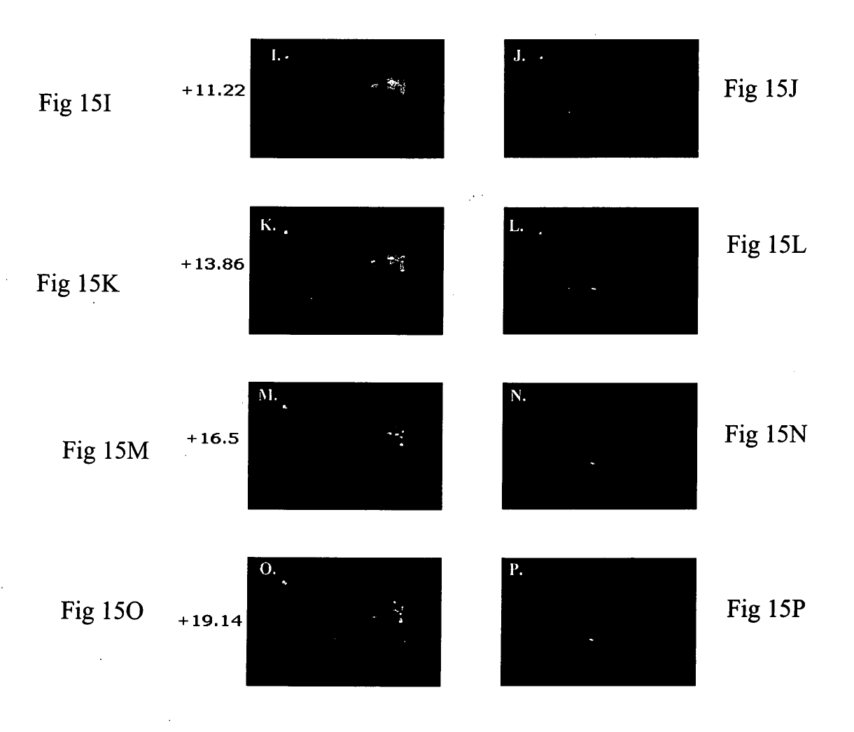

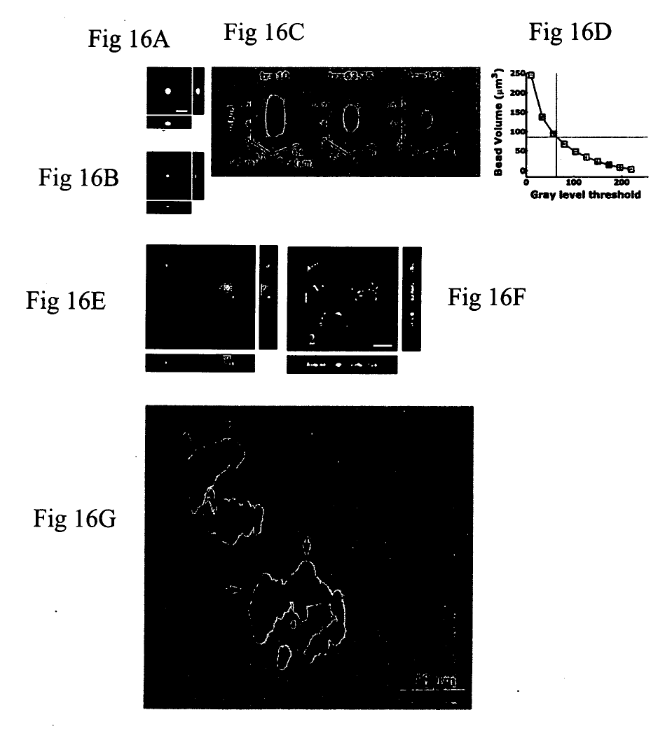

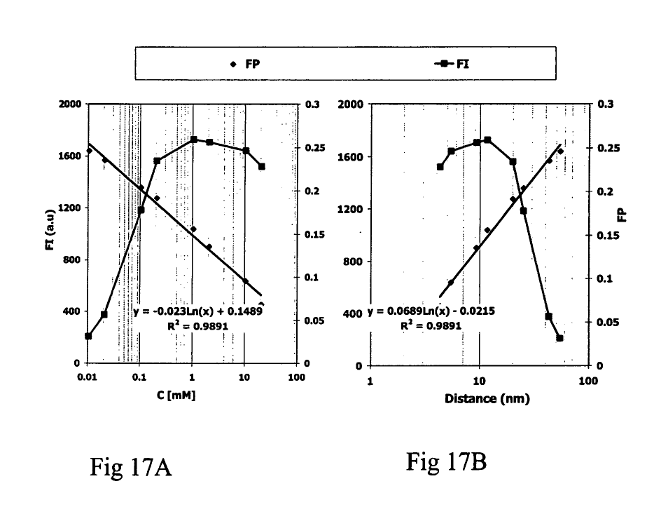

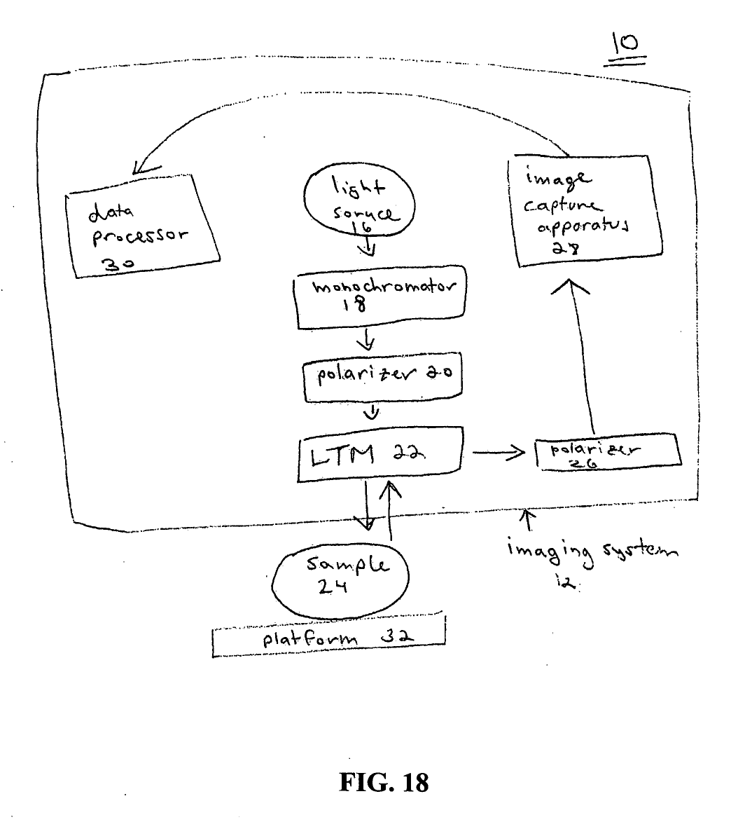

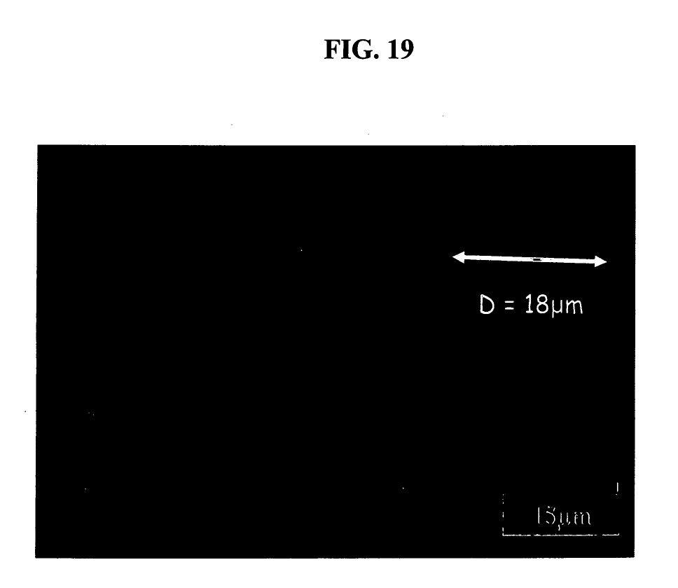

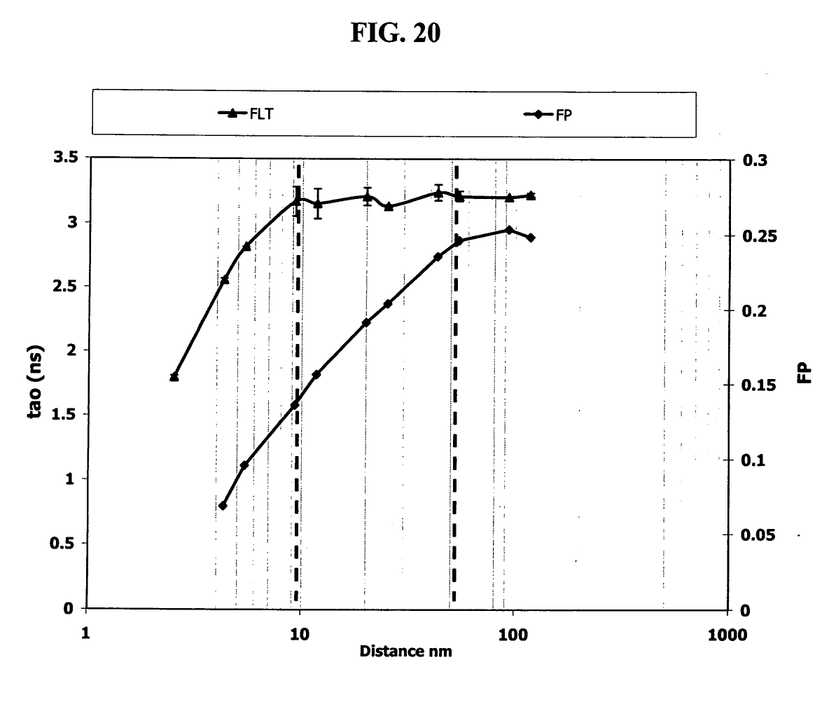

The results obtained in the control cells and ruptured cells are illustrated in FIG. 9. The columns illustrate the average values and standard deviation values for at least 2 identical findings. The stars represent important statistical differences, according to the ANOVA test.

As illustrated in FIG. 9, emission intensity, in every area of the emission, is equal in the two instances following cell rupture. Since the signal is from all the markers, it appears that treatment with CCCP does not diminish the number of fluorescent molecules by disruption or bleaching. The increase in background signal following CCCP treatment alludes to the fact that CCCP does not only cause the probe to leak from the mitochondria to the cytoplasm, but also causes leakage from the entire cell. This finding explains why it appears that the overall signal, measured from the cell (with a microscope or fluocytometer) is low after the treatment, and it answers the question of the seeming absence of the signal.

E2:Nigericin

Materials and Methods

Cell preparation and measurement were conducted using the Cary system, and were identical to those with CCCP, except that Nigericin was used for staining the cells.

Results

The suspension spectra under the different conditions are shown in FIG. 10. Shifts were similar to those represented in FIGS. 8A-B, except for the shift of 2 nm following stimulation with CCCP. This is expected, since in the present test there was no treatment performed for emptying the mitochondria.

The overall emission intensity was calculated as above for CCCP to investigate the cause of the fluorescence intensity increase by calculating the area below the emission graph. The results are presented in FIG. 11. The columns show average values and standard deviation values of at least two identical experiments. The stars indicate the significant statistical differences before the ANOVA test.

As illustrated in FIG. 11, the emission intensity, including the complete emission range, from whole cells treated with Nigericin, is higher than that of untreated cells. This finding indicates that the amount of stain in the cell increases due to the treatment. This conclusion is supported by the fact that the emission intensity, including the entire emission range, from a suspension of ruptured cells, treated prior to rupture with Nigericin, is higher than that received from an untreated cell suspension.

This indicates that the increase in the fluorescence signal is caused by an intensified accumulation of the probe in the cell and not because of changes in its spectroscopic aspects.

F. Influence of Bleaching on Fluorescence Polarization

Probe destruction can influence three variables: intensity, life time and polarization, since it reduces the number of probe molecules. Lifetime is not sensitive to probe decay at low probe concentrations (where the probability of forming dimers is low), but fluorescence polarization is. Therefore, fluorescence polarization of a decaying probe was examined in order to confirm the existence of a homo-interactive mechanism that causes self-depolarization. Intensity was also measured as a control to verify the decay.

Materials and Methods

Cell preparation and measurements were performed as above. Bleaching was induced by exposing cells to excitation light for 5 seconds, and at the end of the exposure cells were imaged. Bleaching was calculated by comparing images prior to and following the bleaching.

The experiment was repeated without bleaching but in the presence of 5 μM CCCP.

The statistical significance of the decay effect on fluorescence polarization, was confirmed by image analysis of over 30 cells.

Results

As illustrated in FIGS. 12A-B, columns show the normalized results (in comparison to the intensity and polarization values of untreated cells) representing corresponding mean and deviation values. In the investigation process, the mitochondria (Mit) and the cytoplasm (Cytoplasm) regions were defined. The columns indicate average intensity and polarization values and, also, standard deviation values which correspond to the entire mitochondria and cytoplasm regions in all the cells. The stars designate the important statistical differences based on the T test.

It can be seen from FIGS. 12A-B that the suppression increased the level of polarization in all the cell regions, but, particularly, in the mitochondrial area. The suppression was greater in the mitochondrial area due to the higher concentration of stain in this section.

When the identical experiment was performed without suppression but with the addition of CCCP, an identical result was obtained—see FIGS. 13A-B. This proves that the mechanism underlying the changes in the polarization of the mitochondria, as a result of stimulation with CCCP, is self-depolarization.

### Example 4

Use of Lifetime Measurements to Monitor Membrane Potential

The following example describes the use of weights f and/or α, decay components, to estimate the probe concentration in the mitochondria and the cytoplasm and from this to derive the membrane potential using the Nernst Equation.

The weight is proportional to the total measured fluorescence signal from an area with a certain fluorescence lifetime. In relying on the results of the measurement, it can be assumed that the decay level in the mitochondria and the cytoplasm is equal and, therefore, is not taken into consideration. In order to convert the weight ratio to concentration ratios, it is necessary to multiply them by the inverted volume ratio, i.e., V_{C}/V_{M} (C-Cytoplasm, M-Mitochondria). Given that this ratio is constant for a certain type of synchronized cells, [data not shown], despite the complex measurement-calculation, it is enough to execute this operation only once.

Materials and Methods

Assessment of the V_{C}/V_{M} Ratio: The mitochondrial volume of U937 cells was calculated from confocal microscopy measurements. The results of the measurements were reinforced in the second stage using convoluted microscopy measurements.

Using a confocal microscope (Zeiss, Germany) a sample of U937 cells, stained with 3 μM Rh123, was excited at a wavelength of 488 nm and sampled at 535±5 nm. After staining and rinsing, 10 μp of cell suspension (350,000/ml) were loaded on a glass cover slip (0.17 mm) which was attached to an aperture that was drilled in the bottom of a Petri dish. In this manner, the necessary distance of the cells from the objective was secured (Plan-Apochromat 63×/1.4 Oil DIC). Laser intensity and exposure time were determined in order to prevent saturation in the detection system. For the given objective data, the best resolution set on the plane X or Y (the plane of the sample) and axis Z, were 0.28 and 0.7 micrometers, respectively.

After each plane scanning, the sample plane was automatically elevated/lowered by steps of 0.7 micrometers, for a total height of 16.8 micrometers, using a Z-Stacks micrometer. The result was 25 measuring planes: half above the focal plane (the plane that passes through the center of the cell) and half below it, in steps of 0.7 micrometers (see FIG. 14A-Y).

This procedure was repeated twice in its entirety, to increase the S/N ratio. The measurement emission results were obtained as an LSM file (Z-Stack file) which can be analyzed using the IPP software.

To evaluate mitochondrial total volume in the cell, their volume was calculated for each plane and integrated over all the planes. The outline of the mitochondria section in each image was determined according to the intensity threshold of 70% from the maximum intensity, based on the assumption that the intensity in the mitochondria is quite uniform. The areas lower than these, were considered to be like those that are adjacent to, but outside of the mitochondria. The calculation, based on 10 cells, yielded an average mitochondrial volume of VM≈5.2 μM³.

Analysis of parameters was performed using the IPP program. First, individual cell boundaries were designated and they were projected on all the planes. Second, it was made certain that there were no deviations from these cell boundaries and the analysis was only in their zone.

With respect to the volume of the whole cell, this was calculated as a spherical volume and its radius was deduced from transmitted light images of the cell. FIG. 19 illustrates a typical example. The calculation yielded an average cell volume of V_{C}=2571 μM³ and therefore, it was concluded that V_{C}/V_{M}≈500.

Measurement of the mitochondrial volume was repeated using a deconvolved microscope to reinforce the above findings. To asses the validity of the deconvolution system, fluorescent beads were imaged (6 micrometer diameter, Molecular Probes manufacture). The step size in axis Z was fixed to be half the field depth of the objective, that is to say, 0.66 μm. In a total result of 45 imaging layers, 12 were chosen to exemplify the measurement process and they are illustrated in FIGS. 15 A-P. The left column of the figure shows the original images which are convolved by the microscope. The right column shows the corrected images, and these are deconvolved, accordingly. The deconvolution was done by means of AutoQuant software, an evaluation system by Imaging Inc. The algorithms were implemented by “blind deconvolution” with parameter values set, as follows: wavelength 517 nm, refractive index n=1, numerical aperture NA=0.6, pixel ratio to real distance in the sample plane of 100 to 16.2 micrometer in plane X-Y and step 0.66 μm along axis Z.

The accuracy of the system was tested in volume assessment. This test was conducted with fluorescent beads after adjusting the spheroid deviation system (see FIGS. 16A-B). Calculating the volume of the bead was performed with a home-developed software kit based on a MATLAB program created by The Mathworks, Inc., MA.

Briefly, the deconvoluted layers were credited with arbitrary threshold intensity values or that in the layers were implemented Otsu algorithms to determine the auto threshold. Three dimensional objects were designed through the integration of voxels (spatial pixels) in conditions of complete three dimensional proximity (Proximity 26). Small objects (less than 4 voxels) were removed from the data evaluation prior to making the volume assessment. The object volume was calculated by multiplying the overall number of voxels, which make up the object, by the size of the calibrated voxel (0.0173 μm³). The dependence of the total voxel volume on the value of the threshold intensity is illustrated in FIGS. 16C-D.

The automatic selection of the threshold intensity by the Otsu system (the junction of red lines in FIG. 16D) produced a calculated volume of 83.435 μm³. This value is only slightly lower than the 84.823 μm³ calculation based on radius 6 μm (the manufacturer's given data). A heuristic system was thus suggested for determining threshold values in other investigations, as well. Caution is necessary, however, when applying this system to continuous objects which have homogenous intensity. Notwithstanding, this practice was implemented before estimation of mitochondria volume, a factor which revealed hidden aspects that were blurred by unfocused light (compare FIGS. 16 E and F). The mitochondrial volume was finally assessed as 7.2 μm³.

The mitochondrial volume calculation based on confocal microscopic findings yielded a similar mitochondrial volume. Consequently, it was decided to continue the research in an arbitrary manner based on the confocal microscopic findings.

Considering the Nernst equation at 300° K and the measured fluorescence signal:

$$Equation 2 \text{:}$$

$$\begin{matrix}Δ Ψ = - \frac{{K}_{B} T}{e} ln \frac{{C}_{M}}{{C}_{C}} ∝ - 26 mV \cdot ln ( \frac{\frac{{α}_{M}}{{V}_{M}}}{\frac{{α}_{C}}{{V}_{C}}} ) \\ = - 26 mV \cdot ln ( \frac{{α}_{M}}{{α}_{C}} \frac{{V}_{C}}{{V}_{M}} )\end{matrix}$$

With respect to the resulting data of the control cells' time measurement (see Table 3):

$$Equation 3 \text{:}$$

$$\begin{matrix}Δ Ψ = - 26 mV \cdot ln ( \frac{{α}_{M}}{{α}_{C}} \frac{{V}_{C}}{{V}_{M}} ) \\ = - 26 mV \cdot ln ( \frac{0.656}{0.314} \cdot 500 ) \\ = - 178.36 mV\end{matrix}$$

This assessment is close to the values measured with other methods [P. Mitchell, J. Moyle, “Estimation of membrane potential and pH difference”, Eur. J. Biochem. 7, 1969, page 47]. Alternately, by evaluating the outcome from the activation with Nigericin, which increases Δψ, the results are:

$$Equation 4 \text{:}$$

$$\begin{matrix}Δ Ψ = - 26 mV \cdot ln ( \frac{{α}_{M}}{{α}_{C}} \frac{{V}_{C}}{{V}_{M}} ) \\ = - 26 mV \cdot ln ( \frac{0.862}{0.138} \cdot 500 ) \\ = - 209.21 mV\end{matrix}$$

It is important to state that, these calculations were made under the supposition that the mitochondrial volume is equal in all the situations, a fact which is not necessarily completely correct. Since these figures correlate with those already known in the literature, it may be assumed that with reference to U937 cells, stained with Rh123 this supposition is correct.

The above correspondence did not occur under the treatment with CCCP. Fitting the corresponding values yielded:

$$Equation 5 \text{:}$$

$$\begin{matrix}Δ Ψ = - 26 mV \cdot ln ( \frac{{α}_{M}}{{α}_{C}} \frac{{V}_{C}}{{V}_{M}} ) \\ = - 26 mV \cdot ln ( \frac{0.310}{0.690} \cdot 500 ) \\ = - 140.8 mV\end{matrix}$$

Indeed, with respect to the f value, a much lower estimation is obtained, approximately Δψ=−112.84 mV. The two outcomes are, of course, higher than the expected outcome. The system is so accurate concerning the two prior conditions, that the latter outcome, raises the question concerning the efficiency of CCCP in these experiments. In fact, an extensive study, reveals that, following stimulation with CCCP, in many cases, the fluorescence intensity remains slightly higher than of the surroundings, which indicates an imbalance of the probe concentration as would be expected under full efficiency of CCCP.

On the other hand, it is also possible that the high concentration in the control state caused a constant absorption (and not specific absorption) of CCCP into the mitochondrial regions, meaning, a non-Nemstian accumulation of the probe. In this case, of course, introduction of measured values into the Nernst equation, will yield Δψ≠0, as expected for this condition. In addition, a new lifetime may be revealed, which similarly will be a shorter one (as was indeed the result, see Table 3). Certainly, the local concentrations of the annexed probe, will, most likely, be higher in such a way that will shorten the lifetime.

Actually, if the system is found to be in pure Nernst it would be plausible to compare concentrations in view of the anticipated activity of CCCP and then the result would be a single lifetime and a single decay component. The significance being the ratio to the index of “decay components” is 1 and the correction factor V_{C}/V_{M}=1. To explain the latter assertion, it must be remembered, this ratio was set for the position in which the mitochondria can be distinguished, meaning, being stained more than their surroundings. Following the regular activation of CCCP, the probe concentration is approximately homogenous, so testing the cell under the confocal microscope would show that the stained volume is approximately that of the entire cell and then the volume ratio would be 1. Of course, applying these numbers in the Nernst equation would yield Δψ≠0. The practical significance of this discussion being, the approach that was proposed is practical as long as it is possible to differentiate between the mitochondria and its environment, that is to say, under the condition that the stain concentration is higher than that of it's surroundings, which brings about the outcome of Δψ≠0.

### Example 5

### Use of Polarization Measurements to Monitor Membrane Potential

As demonstrated in FIG. 20, the “dynamic area” of lifetime measurement is very low and takes place where probe concentration is high. It therefore becomes apparent that not only is there a short dynamic region associated with lifetime measurements, but, that there is no simple way, if at all, to present in a linear manner as dependent on the probe concentration, as was achieved with the polarization. For clarification purposes, FIG. 17A-B comprise the identical data as illustrated in FIG. 20, with the inclusion of intensity data, shown in logarithmic scale.

As stated, from concentrations higher than 0.2 mM, the sensitivity level of the intensity measurement decreases and even the intensity itself decreases, revealing a dependence even in the logarithmic representation. Both the linear and the logarithmic dependence of Rh123 fluorescence polarization on intensity supports the use of such a probe in a monitoring device

Fitting a direct line to the factors in FIG. 16A yields the following equation:

$$Equation 6 \text{:}$$

$$P = - 0.023 ln C + 0.1489 ≡ - a ln C + b$$

$$Equation 7 \text{:}$$

$$Extracting ln C \text{:} ln C = \frac{P - b}{- a}$$

$$Equation 8 \text{:}$$

$$Δ Ψ = - \frac{{K}_{B} T}{e} ln \frac{{C}_{M}}{{C}_{C}} = - \frac{{K}_{B} T}{e} ( ln {C}_{M} - ln {C}_{C} )$$

Now, returning to Nernst's law and the following conclusion:

$$Equation 9 \text{:}$$

$$Sustituting equation 7 in equation 8 for both sites produces \text{:}$$

$$\begin{matrix}Δ Ψ = - \frac{{K}_{B} T}{e} ln \frac{{C}_{M}}{{C}_{C}} \\ = - \frac{{K}_{B} T}{e} ( \frac{{P}_{M} - b}{- a} - \frac{{P}_{C} - b}{- a} ) \\ = \frac{{K}_{B} T}{ea} ( {P}_{M} - {P}_{C} )\end{matrix}$$

$$Equation 10 \text{:}$$

$$Arriving at a different type of connection \text{:}$$

$$Δ Ψ = \frac{{K}_{B} T}{ea} ( {P}_{M} - {P}_{C} )$$

The minus sign in the Nernst equation changes, but the fact is that the polarization in the mitochondria is lower than that in the cytoplasm and, in the end, yields, 0>Δψ; additionally, when the fluorescence polarization of various sites become equal, the result is 0=Δψ. A situation such as this is expected, seemingly, because of stimulation with the CCCP, as already explained.

Testing of the equation's probability was performed, by setting “a” as an absolute value, the result is an refraction index of 26 mv/0.023=1130 mv. Now, fitting two typical polarization values for the mitochondria and the cytoplasm in a control state, for example, 0.150 and 0.320, respectively:

ΔΨ=1130 mV(0.150−0.320)=−192.1 mV Equation 11:

In order to assess Rh123 concentration in U937 cell cytoplasm and mitochondria, 2 ml of Rh123 test suspension (0.013 μM) in PBS, was prepared so the intensity matched that which resulted from a ruptured cell suspension which, prior to being ruptured, contained 7.10⁵ U937 cells stained with Rh123. The number of moles of probe molecules in a test suspension n_{T}, are:

$$Equation 12 \text{:}$$

$$\begin{matrix}{n}_{T} = 2 cc \cdot 0.013 \cdot {10}^{- 6} \frac{mole}{liter} \\ = 2 cc \cdot 0.013 \cdot {10}^{- 6} \frac{mole}{{10}^{3} cc} \\ = 26 \cdot {10}^{- 12} mole\end{matrix}$$

Given that every origin of stain, in the ruptured suspension, is in the cells only, it turns out that the number of probe molecules in Moles and on average, per cell-n, is:

$$Equation 13 \text{:}$$

$$n = \frac{26 \cdot {10}^{- 12} mole}{7 \cdot {10}^{5} cells} = 3.7 \cdot {10}^{- 17} \frac{mole}{cell}$$

Conversely, in relying on transmitted light images, the average volume of U937 cells is:

$$Equation 14 \text{:}$$

$${V}_{cell} ≅ \frac{4 π \cdot {8.5}^{3} μ {m}^{3}}{3} = \frac{4 π \cdot {8.5}^{3} {10}^{- 12} cc}{3} = 257 \cdot {110}^{- 15} L$$

Therefore, the stain concentration in the average cell is:

$$Equation 15 \text{:}$$

$${C}_{cell} = \frac{n}{{V}_{cell}} = \frac{3.7 \cdot {10}^{- 17}}{257 \cdot {110}^{- 15}} M ≅ 14 μ M$$

From the experiments described hereinabove, it can be assumed that 80% of the probe concentrates in the mitochondria. Given that, according to confocal microscopic measurements, the mitochondrial volume in U937 cells are two promil (1/500) of the cell volume, this results in an average probe concentration in the mitochondria in the range of:

C_{M}≈C_{cell}·0.8·500=5.6 mM Equation 16

In closing, the calculations in this section teach, Rh123 stains U937 cell regions in concentrations that range between milimolar to micromolars; At these logarithmic concentration, the inter-molecular distances match the concentrations 5.6 μM and 14 μM and correspond to 66 angstrom and 550 angstrom, respectively.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

What is claimed is:

1. A method of determining a membrane potential, the method comprising:
(a) determining a difference in fluorescence polarization of a charged fluorescent probe being distributed across the membrane; and
(b) determining a potential of the membrane, wherein said potential is proportional to said difference in said fluorescent polarization.

2. The method of claim 1, wherein said potential is linearly proportional to said difference in said fluorescent polarization.

3. The method of claim 1, wherein the membrane is a naturally occurring lipid membrane.

4. The method of claim 1, wherein the membrane is a synthetic membrane.

5. The method of claim 3, wherein the membrane is a cell membrane or an organelle membrane.

6. The method of claim 3, wherein the membrane is a eukaryotic membrane.

7. The method of claim 3, wherein the membrane is a bacterial or viral membrane.

8. The method of claim 6, wherein said eukaryotic membrane is selected from the group consisting of a mammalian membrane, a plant membrane and a fungal membrane.

9. The method of claim 5, wherein said organelle membrane is selected from the group consisting of a mitochondrial membrane, a Golgi membrane and a nuclear membrane.

10. The method of claim 4, wherein said synthetic membrane is a liposome membrane.

11. The membrane of claim 8, wherein said plant membrane is a plastid membrane.

12. The method of claim 11, wherein said plastid membrane is a chloroplast or a leucoplast membrane.

13. The method of claim 1, wherein the membrane is intact.

14. The method of claim 1, wherein said distribution is a steady-state distribution.

15. The method of claim 1, wherein step (a) is effected by generating a map of fluorescence polarization for said distribution of said charged fluorescent probe across the membrane.

16. The method of claim 15, wherein said generating a map is effected by capturing an image of said distribution of said charged fluorescent probe across the membrane and calculating said fluorescence polarization for each picture element of said image.

17. The method of claim 1, wherein said charged fluorescent probe is a cationic dye.

18. The method of claim 1, wherein said charged fluorescent probe is an anionic dye.

19. The method of claim 17, wherein said cationic dye is selected from the group consisting of a Blue-fluorescent SYTO dye, a Green-fluorescent SYTO Dye, an Orange-fluorescent SYTO dye, a Red-fluorescent SYTO dye, SYTO 62, Pur-1, thiazol, aryl, 2DS-7J1, Hoechst 33258, Hoechst 33342 and hexidium iodide.

20. The method of claim 18, wherein said anionic dye is selected from the group consisting of an anionic a bis-isoxazolone oxonol dye, a bis-oxonol dye, Oxonol V, Oxonol VI, DiBAC₄, and DiBAC₂.

21. The method of claim 1, wherein said charged fluorescent probe comprises a fluorescent label selected from the group consisting of rhodamine, tetramethyl rhodamine, carboxytetramethylrhodamine, carboxy-X-rhodamine, fluorescein, fluorinated fluoresceins, fluoresceinamine, carboxyfluorescein, alpha-iodoacetamidofluorescein, 4′-aminomethylfluorescein, 4′-N-alkylaminomethylfluorescein, 5-aminomethylfluorescein, 6-aminomethylfluorescein, 2,4-dichloro-1,3,5-triazin-2-yl-aminofluorescein, 4-chloro-6-methoxy-1,3,5-triazln-2-yl-aminofluorescein, fluoresceinisothiocyanate, 4,4-difluor-4-bora-3a,4a-diaza-s-indacene and its derivatives and cyanine dyes.

22. A system for determining a membrane potential comprising a processing unit, said processing unit executing a software application configured for converting a difference in fluorescence polarization of a charged fluorescent probe being in a distribution across said membrane to a membrane potential.

23. The system of claim 22, further comprising an imaging system.

24. The system of claim 23, wherein said imaging system comprises an image capture apparatus.

25. The system of claim 24, wherein said image capture apparatus is capable of capturing an image of said charged fluorescent probe being in a distribution across said membrane.

26. The system of claim 23, wherein said imaging system further comprises a data processor for calculating said fluorescence polarization for each picture element of said image.

27. The system of claim 26, wherein said data processor is further capable of generating a map of fluorescence polarization for said charged fluorescent probe across the membrane.

28. The system of claim 26, wherein said data processor is further capable of determining an average membrane potential of more than one membrane.

29. The system of claim 24, wherein said image capture apparatus is a fluorescent image capture apparatus and a light image capture apparatus.

30. A method of identifying an agent capable of altering a potential of a membrane, the method comprising:
(a) contacting the membrane with the agent; and subsequently
(b) determining an alteration in the potential of said membrane according to the method of claim 1, wherein a change in said potential following step (b) as compared to a potential of the membrane untreated with the agent is indicative of an agent capable of altering a potential of the membrane.

31. The method of claim 30, wherein said contacting is direct.

32. The method of claim 30, wherein said contacting is indirect.

33. The method of claim 30, wherein the membrane is a cell membrane and/or a mitochondrial membrane.
